SEQUENCE LISTING (9 sequences)

SEQ ID NO: 1 - DNA, 4003 bp, Vibrio harveyi
agctcacggtctttcattgccatacgggaattccatatacagcacatacgcaccagtgcgggtatggcactatcaggtggtgaacgccgccgtgtagaaattgctcgtgcattggcagcaaaccctcagttcattttgttggatgaaccgttcgcgggtgttgacccaatttcggttaacgacatcaaaaaaatcatcgaacacttgcgcgatcgcggccttggcgtgttaatcacagaccataacgtacgcgaaaccttggacgtttgtgaaaaagcctatatcgtaagccaaggacacctcatcgcatcgggaactccggatgaagttctcaataacgagcaagtgaaacaagtttatctcggcgaacaattccgtctatgattacattaggaacggtaagattctgagcattacaaggtaagtaacactgaatgaaaccttcattacaactcaagctaggtcaacagttagccatgacgccacagctgcagcaagcgattcgtttgttgcaattgtcgacgctcgatcttcaacaagaaatccaagaagcgttggactccaacccgctactggaagttgaagaaggccacgatgagcctcaagcaaatggtgaagacaaatcagcgtctgaatctgctgataaaagtgcgaacgaagctaacgatgcctcagaacccgaccttccagatagctcagacgtgattgaaaaatctgaaatcagctctgagctagaaattgataccacttgggatgacgtatatagcgcaaacacgggcagcacaggcctagcgctggatgatgacatgcccgtctaccaaggtgagaccactgaatctttgcatgattaccttatgtggcagttagacttaacgcctttcagtgaaaccgaccgcaccatcgccctcgcgattatcgatgcggtcgacgactacggctacttaaccctatcccctgaagaaattcacgagagcttcgacaacgaagaagtggaattggatgaagtagaagcggtacgtaagcgtattcagcaatttgacccgctcggtgtagcctctcgcaatctgcaagaatgcctactgctacaactggcaactttccctgaagacacgccgtggcttgctgaggcgaaaatggtgttgagcgatcacatcgaccaccttggcaatcgtgactacaagctggtcatcaaagaggctaagcttaaagaagcggacttgcgtgaagtattgaagttgattcaacaacttgacccacgtccaggtagtcgtatcacacccgatgacactgaatacgtcattccggatgtgtccgtatttaaagatcatggtaagtggaccgtctccataaaccctgacagcattccgaaactaaaagtaaatcaacaatatgcgcaactaggcaaaggcaacagtgcggatagccagtacattcgcagcaatttgcaagaggcaaaatggctgattaagagcctagaaagcagaaacgagacgcttctcaaagttgcaagatgtattgttgaacatcaacaagatttcttcgagtatggtgaagaagccatgaaaccaatggtgctaaacgacgtagcattggatgtggacatgcatgaatcgacaatttctcgtgtaacaacacagaagtttatgcataccccacgtggcatttttgaattgaagtacttcttctctagccatgttagtacagacaatggtggagagtgttcgtccacagcaattcgcgcactcatcaaaaagttggtcgcagcggagaataccgctaagccactgagtgatagcaaaattgctgctcttctggctgaccaggggattcaagtcgcgagacggacgatagcaaaatatcgtgaatccttgggtattgccccttcgagtcagcgtaaacgcctactttaggcaccaattgaaaaggaaagtctatgcaaatcaatattcaaggccatcacgttgatcttaccgattcaatgcaagaatatgttgactctaagtttcaaaagctcgagcggttcttcgaccacatcaatcaagtccatgtcgtattaaaagttgaaaaacttaaccaaatagccgaagctacgctccacatcaatcaaggcgaaatccacgcgtcatcgaacgacgaaagtatgtatgcagcaattgattcgctggtggataaattagttcgtcaacttaacaagcacaaagaaaaactaaacagtcattaatcatgcaattgagcgaaatactgtcactggactgcaccaaaagtgcggtccattgtacaagtaagaaacgtgccctcgaaatgatcagccaaattgtcgctgaaaacacgggccaagattctacagaactgtttgagtgtatgctcagcagagaaaaaatgggtagtactggtatcggcaacggtattgctatccctcacgcaagaatgcaatcaagcgacaaagccatcgcagtgttacttcagtgtgacgaagcaattgaatttgacgctatcgacaaccgacctgtcgaccttctttttgctctccttgtacctgaagaacagtgcaaagagcacctcaaaacactatcctctatggcagagcgtctaagtgacaagcaagtgcttaaaagcttacgtaacgctcagagcgatgaagagctctacgacattatgattcataagtaatcaggacgatcaccatgcgattaatcgttgttagcgggcactctggtgccgggaaaagtgttgccctgcgcgtacttgaggacttaggttactactgcgtagacaacctaccggtaaacttacttgacgcgtttgttcagtcagtctctgagagcaaacaaaatgtcgcagtaagcatcgatattcgaaatatccctaagaagctcaaagaactgaataccacgctagagaagctaaaggctgaactggatgtgacagtactgttcttagacgcgaataaagaaacgcttctcacccgctacagcgaaacacgtcggattcatccgctatcacttgacagtcaatcattatcacttgatcaggcgattgagcttgaacaagagatcttaatgcctctgaaagcacacgcagacttagttctgaacagtagcggtcaatctctgcatgatctcagtgaaaccgtacgtatgcgtgtggaaggccgagaacgcaaagacttagtcatggtgtttgagtcgtttggtttcaaatacggtttaccatcagatgccgattacgtgtttgatgtgcgtttcttgccaaacccacactgggagccagcactgcgccctctcactggtttagatggcccgatcggcgccttcttagagcaacaccagtcggtacttgatctgaaataccaaattgaaagctttattgagacttggttaccactattagagaaaaacaaccgtagttacctgaccgttgcgattggttgtactggtggtaaacaccgctcggtttatcttactcaaaaaattggtgagttctttgcggacaaaggacaccaagtacaaattcgccacacttcattggaaaagaacgttaaggaataacggtggaattaagtcgtaaagtactgatccaaaaccgactaggcttgcacgctcgtgcggcagttaaactggtagaactagcacaaagcttcgacgcggtgattaccatcgacaacgaagaagacaaaaccgcgaccgcagacagcgtcatgggattgctgatgctggaatcagcccaaggacaatacgtgaccatccacgccactggcgatcaatctgagcaagctcttgatgcggtttgccatttgatcgaagataagtttgacgaaggcgagtgattcactcgcttttttattatctctagccagatatcccacataagtttcacctcctgcttaaattccgacaaataattttgtcgactttcataagttgttattaaaaggtgcctagaattaagttattattcaaagcattgtaaatatcaggaattgggaggaatgaatggcagagca

SEQ ID NO: 2 - protein, 491 aa, Vibrio harveyi
MKPSLQLKLGQQLAMTPQLQQAIRLLQLSTLDLQQEIQEALDSNPLLEVEEGHDEPQANGEDKSASESADKSANEANDASEPDLPDSSDVIEKSEISSELEIDTTWDDVYSANTGSTGLALDDDMPVYQGETTESLHDYLMWQLDLTPFSETDRTIALAIIDAVDDYGYLTLSPEEIHESFDNEEVELDEVEAVRKRIQQFDPLGVASRNLQECLLLQLATFPEDTPWLAEAKMVLSDHIDHLGNRDYKLVIKEAKLKEADLREVLKLIQQLDPRPGSRITPDDTEYVIPDVSVFKDHGKWTVSINPDSIPKLKVNQQYAQLGKGNSADSQYIRSNLQEAKWLIKSLESRNETLLKVARCIVEHQQDFFEYGEEAMKPMVLNDVALDVDMHESTISRVTTQKFMHTPRGIFELKYFFSSHVSTDNGGECSSTAIRALIKKLVAAENTAKPLSDSKIAALLADQGIQVARRTIAKYRESLGIAPSSQRKRLL

SEQ ID NO: 3 - DNA, 476 bp, Vibrio harveyi
atgaaaccttcattacaactcaagctaggtcaacagttagccatgacgccacagctgcagcaagcgattcgtttgttgcaattgtcgacgctcgatcttcaacaagaaatccaagaagcgttggactccaacccgctactggaagttgaagaaggccacgatgagcctcaagcaaatggtgaagacaaatcagcgtctgaatctgctgataaaagtgcgaacgaagctaacgatgcctcagaacccgaccttccagatagctcagacgtgattgaaaaatctgaaatcagctctgagctagaaattgataccacttgggatgacgtatatagcgcaaacacgggcagcacaggcctagcgctggatgatgacatgcccgtctaccaaggtgagaccactgaatctttgcatgattaccttatgtggcagttagacttaacgcctttcagtgaaaccgaccgcaccatcgccctcgc

SEQ ID NO: 4 - protein, 6 aa, Artificial Sequence
  portion of consensus sequence of sigma-54 domains
XFPGNV

SEQ ID NO: 5 - protein, 6 aa, Artificial Sequence
  portion of consensus sequence of sigma-54 domains
ELFGHX

SEQ ID NO: 6 - DNA, 20 bp, Artificial Sequence
  upstream primer to amplify rpoN gene
ggycaacarttagcsatgac

SEQ ID NO: 7 - DNA, 21 bp, Artificial Sequence
  downstream primer to amplify rpoN gene
catsgcytcytcwccatactc

SEQ ID NO: 8 - DNA, 25 bp, Artificial Sequence
  upstream primer used to amplify rpoN gene
ggaacggtagaattctgagcattac

SEQ ID NO: 9 - DNA, 28 bp, Artificial Sequence
  downstream primer used to amplify rpoN gene
ccttttgaattcgtgcctaaagtaggcg

(19) United States
(12) Patent Application Publication
(10) Pub. No.: US 2003/0023032 A1
(43) Pub. Date: Jan. 30, 2003

(54) LuxO-sigma54 interactions and methods of use

(76) Inventors: Bonnie L. Bassler, Princeton, NJ (US); Brendan N. Lilley, Boston, MA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/853,257

(22) Filed: May 10, 2001

Related U.S. Application Data
(60) Provisional application No. 60/202,999, filed on May 10, 2000.

(51) Int. Cl. C07K 14/705; C07K 16/28; C12P 21/02; C12N 5/06; C07H 21/04

(52) U.S. Cl. 530/350; 530/388.22; 435/69.1; 435/320.1; 435/325; 536/23.5

(57) ABSTRACT

The invention relates to the identification and isolation of a novel sigma 54 (σ⁵⁴) transcription factor from Vibrio harveyi. The invention further relates to the identification of σ⁵⁴ interactions with LuxO. More particularly, the invention provides methods for identifying compounds that regulate bacterial cell growth and virulence by regulating LuxO-σ⁵⁴ activities.

### RELATED APPLICATION INFORMATION

This application claims priority under 35 U.S.C. § 19(e) to U.S. Provisional Application Serial No. 60/202,999, filed May 10, 2000, which is hereby incorporated by reference in its entirety.

### STATEMENT OF GOVERNMENT FUNDED RESEARCH

This work was supported by the National Science Foundation Grant Number MCB-9506033 and The Office of Naval Research Grant Number N00014-99-0767. Accordingly, the Government has certain rights in this invention.

### TECHNICAL FIELD

The invention relates to the identification and isolation of a novel sigma 54 (σ⁵⁴) transcription factor. The invention further relates to the identification of σ⁵⁴ interactions with LuxO. More particularly, the invention provides methods of regulating bacterial cell growth and virulence by regulating LuxO-σ⁵⁴ interactions.

### BACKGROUND

Bacterial pathogenicity can be defined as the molecular mechanisms by which bacteria cause disease. Many bacteria can infect humans or animals, sustain themselves, and multiply on or in host tissues. Disease is an inadvertent but not inevitable consequence of such infection, depending as much on the nature of the host as that of the infecting bacterium. The pathogenicity of bacteria is complex and multifactorial, often involving a series of biochemical mechanisms acting in concert to produce disease. Bacterial virulence factors can be divided broadly into those that assist colonization of the host (e.g. adherence to tissue surfaces and invasion of host cells) and those that assist survival in the hostile environment therein (e.g. resistance to host defenses and the production of toxins).

Intercellular communication is used by bacteria to coordinate colony growth and virulence. The ability to modulate gene expression on a community scale allows bacteria to behave like multi-cellular organisms, and to reap benefits that would otherwise be exclusive to eukaryotes. One type of intercellular communication, termed “quorum sensing” (Bassler, Curr Opin Microbiol 2:582, 1999) was first described in two species of bioluminescent marine bacteria, Vibrio fischeri and Vibrio harveyi (Nealson and Hastings, Microbiol Rev 43:496, 1979). Both bacterial species produce light at high cell population densities which is accomplished through the production of, and response to, extracellular signaling molecules termed autoinducers. In both cases, as the bacteria grow, the concentration of extracellular autoinducer increases. At a critical concentration of autoinducer, a signal transduction cascade is initiated that results in lux expression. Although both Vibrio species use quorum sensing to accomplish the density dependent expression of the luciferase structural operon (luxCDABEG for V. fischeri and luxCDABEGH for V. harveyi), V. fischeri and V. harveyi use different mechanisms for signal production, signal detection, signal relay and signal response (Engebrecht et al., Cell 32:773, 1983; Bassler, In Cell-Cell Signaling in Bacteria, American Society for Microbiology Press, p. 259, 1999).

In V. harveyi, the LuxN/AI-1 quorum sensing circuit is used for intra-species communication, while the LuxPQ/AI-2 quorum sensing circuit is used for inter-species cell-cell signaling, indicating that the two quorum sensing circuits confer on V. harveyi the ability to distinguish self from others. Therefore, V. harveyi monitors not only its own cell-population density but also that of other bacteria. This ability allows V. harveyi to differentially regulate behavior based on whether it exists alone or in consortium. Consistent with this idea, luxs, the gene encoding the AI-2 synthase, is a member of a highly conserved family of genes that specify AI-2 production in a wide range of both Gram negative and Gram positive bacteria. These bacteria include E. coli, S. typhimurium, Salmonella typhi, Vibrio cholerae, Yersinia pestis, Staphylococcus aureus, Streptococcus pyogenes, Enterococcus faecalis, Bacillus subtilis and many others. Thus, AI-2 could be used by some or all of these bacteria for inter-species communication.

LuxP is the primary sensor for AI-2, and the LuxP-AI-2 complex interacts with LuxQ to transmit the autoinducer signal. Signals from both LuxN and LuxQ are channeled to the phosphorelay protein LuxU. LuxU next transmits the signal to the response regulator protein LuxO. Phosphorylation of LuxO activates the protein, and its function is to cause repression of the luxCDABEGH operon. Thus, it would be an advance in the art to identify and characterize the mechanism by which LuxO exerts its effect on downstream expression of various bacterial genes. Such an advance would provide a target for regulating the expression of genes required for bacterial growth and bacterial virulence. Such an advance would further provide a method for identifying compounds that regulate the effect of LuxO for controlling mammalian enteric or pathogenic bacteria growth and virulence.

### SUMMARY

The present invention is based, in part, on the discovery that sigma factor sigma-54 (σ⁵⁴) is required for LuxO function and that, together, LuxO-σ⁵⁴ activate transcription of downstream target genes. The present invention is further based on the identification and isolation a novel σ⁵⁴ transcription factor nucleic acid and protein molecules from Vibrio harveyi. The nucleotide sequence of a cDNA encoding σ⁵⁴ is shown in SEQ ID NO:1, and the amino acid sequence of an σ⁵⁴ polypeptide is shown in SEQ ID NO:2. In addition, the nucleotide sequence of the coding region is depicted in SEQ ID NO:3.

Accordingly, in one aspect, the invention features a nucleic acid molecule that encodes an σ⁵⁴ protein or polypeptide, e.g., a biologically active portion of the σ⁵⁴ protein from Vibrio harveyi. In a preferred embodiment, the isolated nucleic acid molecule encodes a polypeptide having the amino acid sequence of SEQ ID NO:2. In other embodiments, the invention provides isolated σ⁵⁴ nucleic acid molecules having the nucleotide sequence shown in SEQ ID NO: l, SEQ ID NO:3, or the sequence of the DNA insert of the plasmid deposited with ATCC Accession Number AF227983. In still other embodiments, the invention provides nucleic acid molecules that are substantially identical (e.g., naturally occurring allelic variants) to the nucleotide sequence shown in SEQ ID NO: l, SEQ ID NO:3, or the sequence of the DNA insert of the plasmid deposited with ATCC Accession Number AF227983. In other embodiments, the invention provides a nucleic acid molecule that hybridizes under stringent hybridization conditions to a nucleic acid molecule comprising the nucleotide sequence of SEQ ID NO:1 or 3, or the sequence of the DNA insert of the plasmid deposited with ATCC Accession Number AF227983, wherein the nucleic acid encodes a full length σ⁵⁴ protein or an active fragment thereof.

In a related aspect, the invention further provides nucleic acid constructs that include an σ^{54Vh} nucleic acid molecule described herein. In certain embodiments, the nucleic acid molecules of the invention are operatively linked to native or heterologous regulatory sequences. Also included are vectors and host cells containing the σ⁵⁴ nucleic acid molecules of the invention, e.g., vectors and host cells suitable for producing σ⁵⁴ nucleic acid molecules and polypeptides.

In other embodiments, the invention provides σ⁵⁴ polypeptides, e.g., an σ⁵⁴ polypeptide having the amino acid sequence shown in SEQ ID NO:2; the amino acid sequence encoded by the cDNA insert of the plasmid deposited with ATCC Accession Number AF227983; an amino acid sequence that is substantially identical to the amino acid sequence shown in SEQ ID NO:2; or an amino acid sequence encoded by a nucleic acid molecule having a nucleotide sequence that hybridizes under stringent hybridization conditions to a nucleic acid molecule comprising the nucleotide sequence of SEQ ID NO:1 or 3, or the sequence of the DNA insert of the plasmid deposited with ATCC Accession Number AF227983, wherein the nucleic acid encodes a full length σ^{54Vh} protein or an active fragment thereof.

In other embodiments, the invention provides methods for regulating the expression of bacterial genes by regulating the activity of a σ⁵⁴ polypeptide or a LuxO polypeptide. In one aspect, the activity of a σ⁵⁴ polypeptide is regulated by contacting σ⁵⁴ with a LuxO polypeptide. In another aspect, the activity of a LuxO polypeptide is regulated by contacting LuxO with a σ⁵⁴ polypeptide. In another aspect, the activity of a σ⁵⁴ polypeptide is regulated by contacting σ⁵⁴, or LuxO with a compound that regulates σ⁵⁴-LuxO interactions. In a further aspect, the invention provides a method for regulating expression of a bacterial gene by regulating the activity of a σ⁵⁴-LuxO complex.

In another aspect, the invention provides a method for identifying a compound that regulates the binding of a LuxO polypeptide to a σ⁵⁴ polypeptide by contacting a σ⁵⁴ polypeptide with a LuxO polypeptide under conditions and for such time as to allow binding of the σ⁵⁴ polypeptide to the LuxO polypeptide; contacting the σ⁵⁴ polypeptide or LuxO polypeptide of a) with the compound prior to, simultaneously with, or after binding of the σ⁵⁴ polypeptide to the LuxO polypeptide; and measuring the binding of the σ⁵⁴ polypeptide to the LuxO polypeptide in the presence of the compound and comparing it to the binding of the LuxO polypeptide with the σ⁵⁴ polypeptide in the absence of the compound, wherein a change in the binding of a LuxO polypeptide to a σ⁵⁴ polypeptide in the presence of the compound is indicative of a compound that regulates LuxO-σ⁵⁴ binding.

In another aspect, the invention provides a method for identifying a compound that inhibits LuxO-σ⁵⁴ binding by contacting a mixture comprising LuxO and σ⁵⁴ with the compound under conditions and for such time as to allow LuxO-σ⁵⁴ binding; contacting a) with a bacterial cell, or extract thereof, comprising biosynthetic pathways which will produce a detectable amount of light in response to LuxO-σ⁵⁴ binding; and measuring the effect of the compound on light production, wherein decreased light production in the presence of the compound, compared to light production in the absence of the compound, identifies the compound as a compound that inhibits LuxO-σ⁵⁴ binding.

In another aspect, the invention provides a method for identifying a compound that regulates the activity of a LuxO-σ⁵⁴ complex, by contacting a LuxO-σ⁵⁴ complex with the compound; and measuring the activity of the complex in the presence of the compound and comparing the activity of the complex obtained in the presence of the compound to the activity of the complex obtained in the absence of the compound, wherein a change in the activity of the LuxO-σ⁵⁴ complex in the presence of the compound is indicative of a compound that regulates LuxO-σ⁵⁴ complex activity.

In one embodiment, the invention provides a method for regulating expression of a virulence factor in a bacterial cell by contacting a bacterium capable of producing the virulence factor with a compound identified by a method set forth in the present invention. In one aspect, the virulence factor is a siderophore polypeptide. In another aspect, a compound of the invention regulates colony morphology.

In another embodiment, the invention provides a method for treating a subject having a pathogenic bacterial infection by administering to the subject an inhibitor or antagonist that regulates LuxO binding to σ⁵⁴.

In one aspect, the invention provides a method for inhibiting bacterial cell growth or virulence in a subject by administering to the subject an inhibitor or antagonist that regulates LuxO binding to σ⁵⁴.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an alignment of LuxO with other σ⁵⁴ dependent transcriptional activator proteins. Panel A shows an amino acid sequence comparisons between the central portion of LuxO (aa's 134-355) and five other transcriptional activator proteins that interact with σ⁵⁴. These proteins are: NtrC of S. typhimurium (aa's 141-362), NifA of K. pneumoniae (aa's 213-43), DctD of R. leguminosarum (aa's 146-367), HydG of E. coli (aa's 142-363), and FlbD of C. crescentus (aa's 121-342). Amino acids that match the consensus generated for the set of sequences are boxed in black. The glycine rich region that encodes the nucleotide binding domain characteristic of σ⁵⁴-interacting proteins is underlined. Panel B shows a comparison of a C-terminal region of LuxO to that of NtrC, HydG and FlbD. In the box are the putative HTH DNA binding domains for LuxO, HydG and FlbD. The extended box shows the known HTH DNA binding region for NtrC.

FIG. 2 shows the genetic organization of the rpoN region of the V harveyi chromosome. The region of the V. harveyi chromosome that contains the rpoN gene is shown. Sequence analysis indicates that rpoN exists in an operon with at least four other genes. The genetic organization of this region is very similar to that described for the rpoN region of V. cholerae. orf1 is predicted to encode a putative ABC transporter. orf95 is predicted to encode a σ⁵⁴ regulatory protein. ptsN is predicted to encode a nitrogen regulatory protein of the phosphotransferase system and the protein encoded by orf4 has no known function. The locations of the two NsiI sites used to insert a Cm^{r} marker in the construction of the V. harveyi rpoN null mutant are shown and denoted N. R and P denote EcoRI and PstI sites respectively.

FIG. 3 provides photographs indicating σ⁵⁴ is required for motility in V. harveyi, but LuxO. Motility of different V. harveyi strains was assessed using soft-agar plates. The V. harveyi strains to be tested were grown overnight in LM broth, then stabbed into the center of soft-agar LM plates. The plates were incubated for 14 hr at 30° C., after which photographs were taken. Liquid and soft-agar media for V. harveyi strains containing the rpoN::Cm^{r} mutation were supplemented with 1 mM L-glutamine. 10 mg/L Tet was included in broth and soft agar media for the strains carrying plasmid pBNL2090. The V. harveyi strains shown in the figure are: wt, BB120; rpoN::Cm^{r}, BNL240; rpoN::Cm^{r}/prpoN, BNL240/pBNL2090; luxO D47E, JAF548; ΔluxO, JAF78; luxO D47E, rpoN::Cm^{r}, BNL244 and luxO D47E, rpoN::Cm^{r}/prpoN, BNL244/pBNL2090.

FIG. 4 shows that σ⁵⁴ is involved in quorum sensing. Cultures of wild type and mutant V. harveyi strains were grown overnight in AB medium at 30° C. The next day the strains were diluted 5000-fold into fresh AB medium, and light emission was measured every 30 min throughout the subsequent growth of the cultures. Cell density was measured at each time point by diluting the cultures, plating onto LM agar, and counting colonies after overnight growth at 30° C. Symbols: Squares, wild type strain BB120; Triangles, ΔluxO strain JAF78; Circles, rpoN::Cm^{r} null strain BNL240. Relative light units are defined as light emission per cell (i.e., counts min⁻¹ ml⁻¹×10³)/cfu ml⁻¹.

FIG. 5 shows σ⁵⁴ and LuxO regulation of colony morphology in V. harveyi. The smooth and rugose colony morphologies of different V. harveyi strains are shown in the photographs. Each V. harveyi strain was grown in LM broth overnight at 30° C. The strains were streaked onto LM plates, grown for 24 hr at 30° C. and photographed. The strain denotations are the following: wt, BB120; luxO D47E, JAF548; rpoN::Cm^{r}, BNL240 and luxO D47E, rpoN::Cm^{r}, BNL244. Both BNL240 and BNL244 were supplemented with 1 mM L-glutamine in broth and on plates.

FIG. 6 shows LuxO and σ⁵⁴ regulate multiple quorum sensing targets in V. harveyi. The model shows the quorum sensing circuit in V. harveyi. At low cell densities, phosphate flows toward LuxO. Phospho-LuxO is active, and with σ⁵⁴ it activates the transcription of genes required for siderophore production and the rugose colony morphology. The data indicate that LuxO and σ⁵⁴ activate the transcription of an unknown regulatory factor (called X), that negatively regulates the luciferase structural operon luxCDABEGH. Therefore, no light is produced at low cell density. At high cell density, when the autoinducers AI-1 and AI-2 are present, phosphate flows away from LuxO and out of the Lux circuit. Dephosphorylated LuxO is inactive. Therefore, transcription of the genes involved in siderophore production and the rugose colony morphology does not occur. Furthermore, the negative regulator X is not transcribed, so luxCDABEGH is expressed and the bacteria make light. Expression of luxCDABEGH also requires the positive acting factor LuxR. Independently of the quorum sensing circuit, σ⁵⁴ presumably coupled with other transcriptional regulators, controls additional cellular processes in V harveyi. Among these processes are nitrogen metabolism and motility. In the cartoon, H and D denote the conserved His and Asp residues that are the sites of phosphorylation, NT denotes the nucleotide binding/σ⁵⁴ interaction domain, and HTH denotes the Helix-Turn-Helix DNA binding motif.

### DETAILED DESCRIPTION

The present invention provides a novel sigma 54 (σ⁵⁴) transcription factor isolated from V. harveyi and the first identification of a direct interaction between σ⁵⁴ and LuxO. Thus, the invention further provides methods for regulating bacterial cell growth and virulence by regulating LuxO-σ⁵⁴ interactions. The present invention also provides methods for identifying compounds that regulate LuxO-σ⁵⁴ interactions.

Quorum sensing in V. harveyi is mediated by a multi-channel two-component phosphorelay circuit. V. harveyi produces two different autoinducers, AI-1 and AI-2. AI-1 is the acyl-HSL N-3-hydroxybutanoyl-L-homoserine lactone. However, previous reports indicate that AI-2 is not an HSL (Surette and Bassler, Proc Natl Acad Sci USA 95:7046, 1998; Surette and Bassler, Mol Microbiol 31:585, 1999). In V. harveyi, synthesis of AI-1 is dependent on two genes, luxL and luxM, neither of which has homology to the luxI family of autoinducer synthases. Similarly, synthesis of AI-2 is dependent on the gene luxS, which also shows no homology to luxI. Detection of AI-1 and AI-2 occurs via the cognate sensors LuxN and LuxPQ, respectively. LuxN and LuxQ are two-component hybrid sensor kinases containing both a sensor kinase domain and an attached response regulator domain. LuxP is homologous to the ribose binding protein of Escherichia coli and Salmonella typhimurium. These studies indicate that LuxP is the primary sensor for AI-2, and that the LuxP-AI-2 complex interacts with LuxQ to transmit the autoinducer signal. Signals from both LuxN and LuxQ are channeled to the phosphorelay protein LuxU. LuxU next transmits the signal to the response regulator protein LuxO.

At low cell density and in the absence of autoinducer, the LuxN and LuxQ sensors act as kinases. The sensors autophosphorylate on conserved His residues and transfer the phosphoryl group to the conserved Asp residues in their attached response regulator domains. Thus, the first phosphotransfer event is intra-molecular. Subsequently, inter-molecular phospho-transfer occurs from both sensors to the conserved His residue of the phosphorelay protein LuxU. In the final step, the phosphoryl group is transferred to the conserved Asp in the response regulator protein LuxO. Phosphorylation of LuxO activates the protein, and its function is to cause repression of the luxCDABEGH operon. Therefore, at low cell density, the bacteria make no light. At high cell density and in the presence of their cognate autoinducers, LuxN and LuxQ alter their activities, and switch from being kinases to being phosphatases. In this mode, the sensors drain phosphate out of the system. The phosphatase activities of the sensors result in rapid elimination of LuxO-phosphate, and the dephosphorylated form of LuxO is inactive. Therefore, at high cell density, no repression of luxCDABEGH occurs, and the bacteria emit light. A transcriptional activator called LuxR, that is not related to LuxR from V fischeri, is also required for the expression of the luxCDABEGH operon in V. harveyi.

The present invention provides an isolated nucleic acid encoding a novel σ⁵⁴ polypeptide from V. harveyi. The present invention also shows for the first time that σ⁵⁴ interacts with the response regulator protein LuxO. The interaction of σ⁵⁴ with LuxO provides a target for regulating bacterial quorum sensing system I or II. In turn, the regulation of bacterial quorum sensing provides a mechanism for regulating bacterial growth and pathogenesis. In addition, the interaction of σ⁵⁴ with LuxO provides mechanism for identifying compounds that regulate the LuxO-σ⁵⁴ interaction or compounds that regulate the activity of a LuxO-σ⁵⁴ complex.

σ⁵⁴ Nucleic Acid, Polypeptides, Host Cells and Vectors

In one embodiment, the invention provides an isolated polynucleotide sequence encoding a σ⁵⁴ polypeptide from V. harveyi. An exemplary σ⁵⁴ polypeptide of the invention has an amino acid sequence as set forth in SEQ ID NO:2. The term “isolated” as used herein includes polynucleotides substantially free of other nucleic acids, proteins, lipids, carbohydrates or other materials with which it is naturally associated. Polynucleotide sequences of the invention include DNA and RNA sequences which encode σ⁵⁴. It is understood that all polynucleotides encoding all or a portion of σ⁵⁴ are also included herein, as long as they encode a polypeptide with σ⁵⁴ activity. Such polynucleotides include naturally occurring, synthetic, and intentionally manipulated polynucleotides. For example, σ⁵⁴ polynucleotide may be subjected to site-directed mutagenesis. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of σ⁵⁴ polypeptide encoded by the nucleotide sequence is functionally unchanged. Also included are nucleotide sequences which encode σ⁵⁴ polypeptide, such as SEQ ID NO:1. In addition, the invention also includes a polynucleotide encoding a polypeptide having the biological activity of an amino acid sequence of SEQ ID NO:2 and having at least one epitope for an antibody immunoreactive with σ⁵⁴ polypeptide.

The invention includes polypeptides having substantially the same amino acid sequence as set forth in SEQ ID NO:2 or functional fragments thereof, or amino acid sequences that are substantially identical to SEQ ID NO:2. By “substantially the same” or “substantially identical” is meant a polypeptide or nucleic acid exhibiting at least 80%, preferably 85%, more preferably 90%, and most preferably 95% homology to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

By “substantially identical” is also meant an amino acid sequence which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein assayed, (e.g., as described herein). Preferably, such a sequence is at least 85%, more preferably identical at the amino acid level to SEQ ID NO:2.

Homology is often measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various substitutions, deletions, substitutions, and other modifications.

By a “substantially pure polypeptide” is meant an σ⁵⁴ polypeptide which has been separated from components which naturally accompany it. Typically, the polypeptide is substantially pure when it is at least 60%, by weight, free from the proteins and naturally occurring organic molecules with which it is naturally associated. Preferably, the preparation is at least 75%, more preferably at least 90%, and most preferably at least 99%, by weight, σ⁵⁴ polypeptide. A substantially pure σ⁵⁴ polypeptide may be obtained, for example, by extraction from a natural source (e.g., a plant cell); by expression of a recombinant nucleic acid encoding an σ⁵⁴ polypeptide; or by chemically synthesizing the protein. Purity can be measured by any appropriate method, e.g., those described in column chromatography, polyacrylamide gel electrophoresis, or by HPLC analysis.

σ⁵⁴ polypeptides of the present invention include peptides, or full-length protein, that contains substitutions, deletions, or insertions into the protein backbone, that would still leave a 70% homology to the original protein over the corresponding portion. A yet greater degree of departure from homology is allowed if like-amino acids, i.e. conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutainine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

The polynucleotide encoding σ⁵⁴ includes the nucleotide sequence in SEQ ID NO:1, as well as nucleic acid sequences complementary to that sequence. When the sequence is RNA, the deoxyribonucleotides A, G, C, and T of SEQ ID NO:1 are replaced by ribonucleotides A, G, C, and U, respectively. Also included in the invention are fragments (portions) of the above-described nucleic acid sequences that are at least 15 bases in length, which is sufficient to permit the fragment to selectively hybridize to DNA that encodes the protein of SEQ ID NO:2. “Selective hybridization” as used herein refers to hybridization under moderately stringent or highly stringent physiological conditions (See, for example, the techniques described in Maniatis et al., 1989 Molecular Cloning A Laboratory Manual, Cold Spring Harbor Laboratory, N.Y., incorporated herein by reference), which distinguishes related from unrelated nucleotide sequences.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2× SSC/0.1% SDS at about room temperature (hybridization conditions); 0.2× SSC/0.1% SDS at about room temperature (low stringency conditions); 0.2× SSC/0.1% SDS at about 42EC (moderate stringency conditions); and 0.1× SSC at about 68EC (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Primers used according to the method of the invention are designed to be “substantially” complementary to each strand of mutant nucleotide sequence to be amplified. Substantially complementary means that the primers must be sufficiently complementary to hybridize with their respective strands under conditions that allow the agent for polymerization to function. In other words, the primers should have sufficient complementarily with the flanking sequences to hybridize therewith and permit amplification of the mutant nucleotide sequence. Preferably, the 3′ terminus of the primer that is extended has perfectly base paired complementarity with the complementary flanking strand.

DNA sequences encoding V. harveyi σ⁵⁴ can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, the σ⁵⁴ polynucleotide sequences may be inserted into a recombinant expression vector. The term “recombinant expression vector” refers to a plasmid, virus or other vehicle known in the art that has been manipulated by insertion or incorporation of the σ⁵⁴ genetic sequences. Such expression vectors contain a promoter sequence that facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al., Gene ,56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein I, or polyhedrin promoters).

Polynucleotide sequences encoding V. harveyi σ⁵⁴ can be expressed in either prokaryotes or eukaryotes. Hosts can include microbial, yeast, insect and mammalian organisms. Such vectors are used to incorporate DNA sequences of the invention.

Methods that are well known to those skilled in the art can be used to construct expression vectors containing the σ⁵⁴ coding sequence and appropriate transcriptional/translational control signals. These methods include in vitro recombinant DNA techniques, synthetic techniques, and in vivo recombination/genetic techniques. (See, for example, the techniques described in Maniatis et al., 1989 Molecular Cloning A Laboratory Manual, Cold Spring Harbor Laboratory, N.Y.)

A variety of host-expression vector systems may be utilized to express the S54 coding sequence. These include but are not limited to microorganisms such as bacteria transformed with recombinant bacteriophage DNA, plasmid DNA or cosmid DNA expression vectors containing the σ⁵⁴ coding sequence; yeast transformed with recombinant yeast expression vectors containing the σ⁵⁴ coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the σ⁵⁴ coding sequence; insect cell systems infected with recombinant virus expression vectors (e.g., baculovirus) containing the σ⁵⁴ coding sequence; or animal cell systems infected with recombinant virus expression vectors (e.g. retroviruses, adenovirus, vaccinia virus) containing the σ⁵⁴ coding sequence, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (see e.g., Bitter et al., Methods in Enzymology 153:516, 1987). For example, when cloning in bacterial systems, inducible promoters such as pL of bacteriophage (, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the inserted σ⁵⁴ coding sequence.

In yeast, a number of vectors containing constitutive or inducible promoters may be used. For a review, see Current Protocols in Molecular Biology, Vol. 2, 1988, Ed., Ausubel et al, Greene Publish. Assoc. & Wiley Interscience, Ch. 13; Grant et al., Expression and Secretion Vectors for Yeast, in Methods in Enzymology, 153:516, 1987; Glover, 1986, DNA Cloning, Vol. II, IRL Press, Wash., D.C., Ch. 3; and Bitter, Heterologous Gene Expression in Yeast, Methods in Enzymology, 152 673, 1987; and The Molecular Biology of the Yeast Saccharomyces, 1982, Eds. Strathem et al., Cold Spring Harbor Press, Vols. I and II. A constitutive yeast promoter such as ADH or LEU2 or an inducible promoter such as GAL may be used (Cloning in Yeast, Ch. 3, R. Rothstein In: DNA Cloning Vol.11, A Practical Approach, Ed. D M Glover, 1986, IRL Press, Wash., D.C.). Alternatively, vectors may be used which promote integration of foreign DNA sequences into the yeast chromosome.

The genetic construct can be designed to provide additional benefits, such as, for example addition of C-terminal or N-terminal amino acid residues that would facilitate purification by trapping on columns or by use of antibodies. All those methodologies are cumulative. For example, a synthetic gene can later be mutagenized. The choice as to the method of producing a particular construct can easily be made by one skilled in the art based on practical considerations: size of the desired peptide, availability and cost of starting materials, etc. All the technologies involved are well established and well known in the art. See, for example, Ausubel et al., Current Protocols in Molecular Biology, Volumes 1 and 2 (1987), with supplements, and Maniatis et al., Molecular Cloning, a Laboratory Manual, Cold Spring Harbor Laboratory (1989). Yet other technical references are known and easily accessible to one skilled in the art.

Antibodies that Bind to σ⁵⁴

In another embodiment, the present invention provides antibodies that bind to σ⁵⁴. Such antibodies are useful for research and diagnostic tools in the study of bacterial infection in general, and specifically the development of more effective anti-bacterial therapeutics. Such antibodies may be administered alone or contained in a pharmaceutical composition comprising antibodies against σ⁵⁴ and other reagents effective as anti-bacterial therapeutics.

Antibodies that bind to the σ⁵⁴ polypeptide of the invention can be prepared using an intact polypeptide or fragments containing small peptides of interest as the immunizing antigen. For example, one of skill in the art can use the peptides to generate appropriate antibodies of the invention. Antibodies of the invention include polyclonal antibodies, monoclonal antibodies, and fragments of polyclonal and monoclonal antibodies.

The preparation of polyclonal antibodies is well known to those skilled in the art. See, for example, Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, section 2.4.1 (1992), which are hereby incorporated by reference.

The preparation of monoclonal antibodies likewise is conventional. See, for example, Kohler & Milstein, Nature 256:495 (1975); Coligan et al., sections 2.5.1-2.6.7; and Harlow et al., Antibodies: A Laboratory Manual, page 726 (Cold Spring Harbor Pub. 1988), which are hereby incorporated by reference. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., Purification of Immunoglobulin G (IgG), in Methods in Molecular Biology, Vol. 10, pages 79-104 (Humana Press 1992). Methods of in vitro and in vivo multiplication of monoclonal antibodies is well known to those skilled in the art. Multiplication in vitro may be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., osyngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Compounds

In another embodiment, the invention provides a method for identifying a compound that modulates a LuxO-σ⁵⁴ interaction. The invention further provides a method for identifying a compound that modulates the activity of a LuxO-σ⁵⁴ complex. The method includes: a) incubating components comprising the compound in the presence of LuxO, σ⁵⁴, or LuxO and σ⁵⁴ under conditions sufficient to allow the components to interact; and b) determining the effect of the compound on LuxO, σ⁵⁴, or LuxO and σ⁵⁴ activity before and after incubating in the presence of the compound. Compounds that affect LuxO, ^{σ}⁵⁴, or LuxO and σ⁵⁴ activity include peptides, peptidomimetics, polypeptides, chemical compounds and biologic agents. The invention further provides methods for identifying a compound that regulates the activity of a LuxO-σ⁵⁴ complex.

Incubating includes conditions that allow contact between the test compound and LuxO, σ⁵⁴, or LuxO and σ⁵⁴ or a LuxO-σ⁵⁴ complex. Contacting includes in solution and in solid phase, or in a cell. The test compound may optionally be a combinatorial library for screening a plurality of compounds. Compounds identified in the method of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3:1008-1012, 1985), allele-specific oligonucleotide (ASO) probe analysis (Conner, et al., Proc. Natl. Acad. Sci. USA, 80:278, 1983), oligonucleotide ligation assays (OLAS) (Landegren, et al., Science, 241:1077, 1988), and the like. Molecular techniques for DNA analysis have been reviewed (Landegren, et al., Science, 242:229-237, 1988).

Thus, the method of the invention includes combinatorial chemistry methods for identifying chemical compounds that bind to LuxO, σ⁵⁴, or LuxO and σ⁵⁴ or a LuxO-σ⁵⁴ complex or affect the activity of LuxO, σ⁵⁴, or LuxO and σ⁵⁴ or a LuxO-σ⁵⁴ complex. By identifying an interaction between LuxO and σ⁵⁴ the invention provides a means for identifying ligands or substrates that bind to, modulate, affect the expression of, or mimic the action of an LuxO, σ⁵⁴, or LuxO and σ⁵⁴ or a LuxO-σ⁵⁴ complex.

Areas of investigation are the development of therapeutic treatments. The screening identifies compounds that provide regulation of LuxO, σ⁵⁴, or LuxO and σ⁵⁴ or a LuxO-σ⁵⁴ complex function in targeted microorganisms. Of particular interest are screening assays for compounds that have a low toxicity for humans. A wide variety of assays may be used for this purpose, including labeled in vitro protein-protein binding assays, protein-DNA binding assays, electrophoretic mobility shift assays, immunoassays for protein binding, and the like. The purified protein may also be used for determination of three-dimensional crystal structure, which can be used for modeling intermolecular interactions and transcriptional regulation, for example.

The term “compound” as used herein describes any molecule or agent, e.g. protein or pharmaceutical, with the capability of regulating, altering or mimicking the physiological function or expression of an LuxO, σ⁵⁴, or LuxO and σ⁵⁴ or a LuxO-σ⁵⁴ complex. Generally, a plurality of assay mixtures are run in parallel with different compound concentrations to obtain a differential response to the various concentrations. Typically, one of these concentrations serves as a negative control, i.e. at zero concentration or below the level of detection.

Candidate compounds encompass numerous chemical classes, though typically they are organic molecules, preferably small organic compounds having a molecular weight of more than 50 and less than about 2,500 daltons. Candidate compounds comprise functional groups necessary for structural interaction with proteins, particularly hydrogen bonding, and typically include at least an amine, carbonyl, hydroxyl or carboxyl group, preferably at least two of the functional chemical groups. The candidate compounds often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof. Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification and amidification to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors and anti-microbial agents may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

The invention further provides methods for identifying a compound that binds to a protein of the invention, such as LuxO or σ⁵⁴, or a LuxO-σ⁵⁴ complex. The method includes incubating components comprising the compound and LuxO or σ⁵⁴, or a LuxO-σ⁵⁴ complex, under conditions sufficient to allow the components to interact and measuring the binding of the compound to LuxO or σ⁵⁴, or a LuxO-σ⁵⁴ complex. Compounds that bind to LuxO or σ⁵⁴, or a LuxO-σ⁵⁴ complex, include peptides, peptidomimetics, polypeptides, chemical compounds and biologic agents as described above.

Incubating includes conditions that allow contact between the test compound and LuxO or σ⁵⁴, or a LuxO-σ⁵⁴ complex. Contacting includes in solution and in solid phase. The test ligand(s)/compound may optionally be a combinatorial library for screening a plurality of compounds. Compounds identified in the method of the invention can be further evaluated, detected, cloned, sequenced, and the like, either in solution or after binding to a solid support, by any method usually applied to the detection of a specific DNA sequence such as PCR, oligomer restriction (Saiki et al., Bio/Technology, 3:1008-1012, 1985), allele-specific oligonucleotide (ASO) probe analysis (Conner et al., Proc. Natl. Acad. Sci. USA, 80:278, 1983), oligonucleotide ligation assays (OLAs) (Landegren et al., Science, 241:1077, 1988), and the like. Molecular techniques for DNA analysis have been reviewed (Landegren et al., Science, 242:229-237, 1988). Also included in the screening method of the invention are combinatorial chemistry methods for identifying chemical compounds that bind to LuxP or LuxQ. See, for example, Plunkett and Ellman, “Combinatorial Chemistry and New Drugs,” Scientific American, April, p.69 (1997).

Thus, the present invention to provide compounds and methods for regulating the effect of LuxO-σ⁵⁴ activity on expression of downstream genes. Provided herein are pharmaceutical compositions comprising such compounds and methods of using the compounds and compositions of the invention to regulate bacterial growth and virulence by regulating the activity of LuxO-σ⁵⁴ activity and proteins that interact with LuxO or σ⁵⁴, or a LuxO-σ⁵⁴ complex. Thus, the invention provides a mechanism for the control of bacterial growth, such as by inhibition of bacterial growth, utilizing the compounds of the invention. The invention further provides a mechanism to not only control bacterial growth but also to control those pathways involved in expression of phenotypes associated with bacterial virulence and pathogenicity such as siderophore production and rugose polysaccharide production.

Quorum sensing is a major regulator of biofilm control and quorum-sensing blockers can therefore be used to prevent and/or inhibit biofilm formation. Also, quorum-sensing blockers are effective in removing, or substantially decreasing, the amount of biofilms that have already formed on a surface. Thus, by determining that a σ⁵⁴-LuxO interaction regulates the expression of bacterial genes, the present invention provides a new approach to inhibiting bacterial infections by identifying compounds that regulate the activity of LuxO-σ⁵⁴ interactions. Such compounds can be used to regulate biofilm formation and can be included in a pharmaceutical composition as described in the present specification.

In another embodiment, the invention provides a method of removing a biofilm from a surface that comprises treating the surface with a compound identified by a method of the invention. The surface is preferably the inside of an aqueous liquid distribution system, such as a drinking water distribution system or a supply line connected to a dental air-water system. The removal of biofilms from this type of surface can be particularly difficult to achieve. The compound is preferably applied to the surface as a solution of the compound either alone or together with other materials such as conventional detergents or surfactants.

A further embodiment of the invention is an antibacterial composition comprising a compound of the invention together with a bacteriocidal agent. In the antibacterial compositions, the compound of the invention helps to remove the biofilm whilst the bacteriocidal agent kills the bacteria. The antibacterial composition is preferably in the form of a solution or suspension for spraying and/or wiping on a surface.

In yet another aspect, the invention provides an article coated and/or impregnated with a compound of the invention in order to inhibit and/or prevent biofilm formation thereon. The article is preferably of plastics material with the compound of the invention distributed throughout the material.

Pharmaceutical Compositions

The invention further provides pharmaceutical compositions for preventing or treating pathogen-associated diseases by targeting factors involved in the Signaling System type-2 pathway. A pharmaceutical composition of the invention can include a compound that regulates the activity of LuxO, σ⁵⁴, or LuxO and σ⁵⁴ or a LuxO-σ⁵⁴ complex. For example, the present invention provides information that LuxO is associated with siderophore production and exopolysaccharide production in a bacterial cell. The activity of LuxO is directly related to the activity of σ⁵⁴. Thus, compounds that regulate LuxO activity, for example, will also regulate σ⁵⁴ activity and effect the expression of a virulence factor, such as siderophore or exopolysaccharide production. The present invention clearly provides a mechanism for regulating biochemical pathways controlled by LuxO and σ⁵⁴ activity by providing identifying an interaction between LuxO and σ⁵⁴.

In addition, LuxO, σ⁵⁴, or LuxO and σ⁵⁴ or a LuxO-σ⁵⁴ complex provide a common target for the development of a vaccine. Antibodies raised to LuxO or σ⁵⁴, or a LuxO-σ⁵⁴ complex, or homologs thereof, can inhibit the activation of bacterial pathways associated with virulence. Thus, LuxO and σ⁵⁴ provide common antigenic determinants that can be used to immunize a subject against multiple pathogen-associated disease states. For example, the autoinducer Signaling System type-2 is believed to exist in a broad range of bacterial species including bacterial pathogens. As discussed above, the autoinducer-2 signaling factor is believed to be involved in inter-species as well as intra-species communication. In order for the quorum-sensing Signaling System type-2 to be effective for inter-species communication, it is likely to be highly conserved among various bacterial species. Thus, challenging a subject with the LuxO and σ⁵⁴ polypeptide, or an antigenic fragment thereof, isolated from a particular organism may confer protective immunity to other disease states associated with a different organism.

Generally, the terms “treating”, “treatment”, and the like are used herein to mean obtaining a desired pharmacologic and/or physiologic effect. The effect may be prophylactic in terms of completely or partially preventing a microbial infection or disease or sign or symptom thereof, and/or may be therapeutic in terms of a partial or complete cure for an infection or disease and/or adverse effect attributable to the infection or disease. “Treating” as used herein covers any treatment of (e.g., complete or partial), or prevention of, an infection or disease in a mammal, particularly a human, and includes:

(a) preventing the disease from occurring in a subject that may be predisposed to the disease, but has not yet been diagnosed as having it;

(b) inhibiting the infection or disease, i.e., arresting its development; or

(c) relieving or ameliorating the infection or disease, i.e., cause regression of the infection or disease.

Thus, the invention includes various pharmaceutical compositions useful for ameliorating symptoms attributable to a bacterial infection or, alternatively, for inducing a protective immune response to prevent such an infection. For example, a pharmaceutical composition according to the invention can be prepared to include a compound that regulates LuxO binding to σ⁵⁴ or regulates the activity of a LuxO-σ⁵⁴ complex such that bacterial cell growth is regulated or the expression of a virulence factor is regulated. The pharmaceutical composition can further include a binding compound according to the present invention into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th Ed.).

The pharmaceutical compositions according to the invention may be administered locally or systemically. By “therapeutically effective dose” is meant the quantity of a compound according to the invention necessary to prevent, to cure or at least partially arrest the symptoms of the disease and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

As used herein, “administering a therapeutically effective amount” is intended to include methods of giving or applying a pharmaceutical composition of the invention to a subject that allow the composition to perform its intended therapeutic function. The therapeutically effective amounts will vary according to factors such as the degree of infection in a subject, the age, sex, and weight of the individual. Dosage regima can be adjusted to provide the optimum therapeutic response. For example, several divided doses can be administered daily or the dose can be proportionally reduced as indicated by the exigencies of the therapeutic situation.

The pharmaceutical composition can be administered in a convenient manner such as by injection (subcutaneous, intravenous, etc.), oral administration, inhalation, transdermal application, or rectal administration. Depending on the route of administration, the pharmaceutical composition can be coated with a material to protect the pharmaceutical composition from the action of enzymes, acids and other natural conditions that may inactivate the pharmaceutical composition. The pharmaceutical composition can also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle which contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5 to about 80% of the weight of the unit. The amount of pharmaceutical composition in such therapeutically useful compositions is such that a suitable dosage will be obtained.

The tablets, troches, pills, capsules and the like can also contain the following: a binder such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the agent, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

As used herein, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete units suited as unitary dosages for the individual to be treated; each unit containing a predetermined quantity of pharmaceutical composition is calculated to produce the desired therapeutic effect in association with the required pharmaceutical carrier. The specification for the novel dosage unit forms of the invention are dictated by and directly dependent on (a) the unique characteristics of the pharmaceutical composition and the particular therapeutic effect to be achieve, and (b) the limitations inherent in the art of compounding such an pharmaceutical composition for the treatment of a pathogenic infection in a subject.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

Results

As previously noted, two signal-response systems control quorum sensing in V. harveyi. Each system is composed of an autoinducer/two-component sensor pair (AI-1/LuxN and AI-2/LuxPQ). Signaling from both two-component sensors converges at a shared phosphorelay protein called LuxU. Finally LuxU transfers signal to the response regulator protein LuxO. Phospho-LuxO is responsible for repression of the expression of the luciferase structural operon luxCDABEGH at low cell densities and low autoinducer concentrations.

LuxO is a homologue of NtrC and it contains each of the conserved domains (response regulator, σ⁵⁴ activation, helix-turn-helix DNA binding) present in NtrC and other transcriptional activators that work in concert with σ⁵⁴. These results indicate that LuxO is also a σ⁵⁴-dependent transcriptional activator. However, the role of LuxO in the V. harveyi quorum sensing system is to cause repression of lux expression at low cell density. Consistent with this, some members of the NtrC family of proteins possess both activator and repressor activities. For example, in C. crescentus, phospho-FlbD, together with σ⁵⁴, activates the expression of class III flagellar genes. However, FlbD also represses transcription of the fliF operon in a manner that is partially dependent upon the phosphorylation state of FlbD. Unlike the activation function of FlbD, repressor function is not dependent on σ⁵⁴. As discussed above, in S. typhimurium, phospho-NtrC, in conjunction with σ⁵⁴, activates transcription of glnA. NtrC also represses transcription of a minor σ⁷⁰ promoter that is upstream of the major glnA σ⁵⁴-regulated promoter. As in the case of FlbD, σ⁵⁴ is required for the activation function of NtrC but it is not required for the repressor activity.

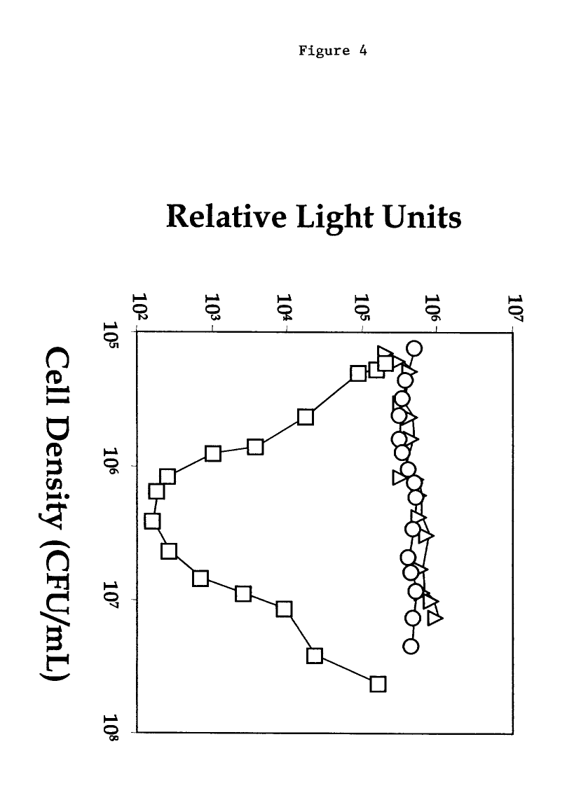

In the present study the rpoN gene (encoding σ⁵⁴) from V. harveyi has been cloned, analyzed and mutated. The phenotype of a V. harveyi rpoN null mutant was constructed and the results indicate that it does not express luminescence in a density dependent manner (FIG. 4). Rather, it exhibits maximal, constitutive expression of luminescence. The phenotype of the rpoN mutant is indistinguishable from that of a luxO null mutant strain. This result demonstrates that both LuxO and σ⁵⁴ are required for repression of the expression of luminescence at low cell density. The present study further shows that the function of LuxO in the Lux quorum sensing circuit is dependent on σ⁵⁴ (Table 1). The fact that LuxO requires σ⁵⁴ for repression indicates that LuxO is an activator not a repressor. The data further indicates that, in the Lux circuit, phospho-LuxO and σ⁵⁴ activate the transcription of an unknown factor that is the true repressor of luxCDABEGH.

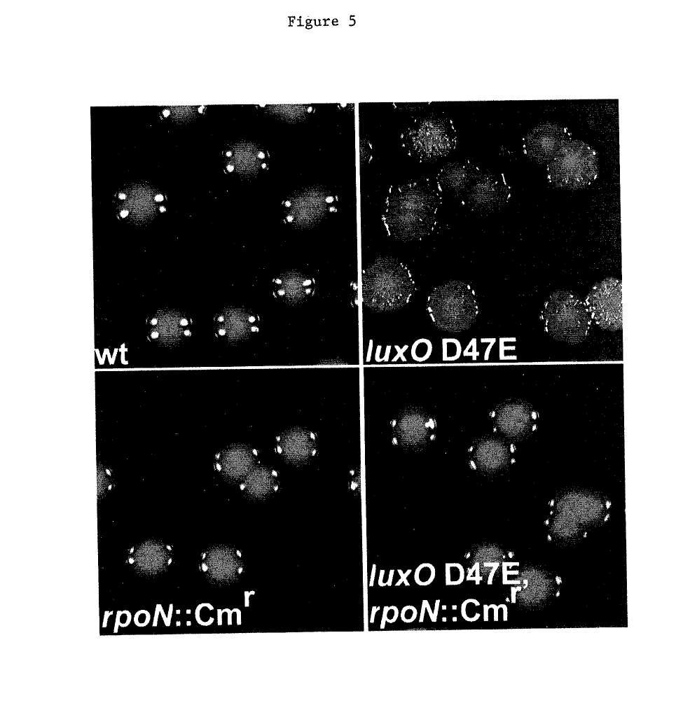

The present study indicates that siderophore production and colony morphology phenotypes are also under the control of LuxO and σ⁵⁴ (Table 3 and FIG. 5). These are the first examples of quorum sensing regulated phenotypes, other than Lux, in V. harveyi.

Regulation of siderophore production in many species of bacteria including E. coli and V. cholerae is under the control of the ferric uptake regulation (Fur) protein. In these cases, under iron rich conditions, the Fur protein binds Fe²⁺ ions and represses the transcription of genes required for siderophore biosynthesis and transport. De-repression of these genes occurs during periods of iron deprivation, when Fur is not bound to Fe²⁺. The results presented in Table 3 indicate that LuxO and σ⁵⁴ have a role in activating the production of siderophore in V. harveyi. However, neither LuxO nor σ⁵⁴ is necessary for growth on medium containing the iron chelator EDDA. A Fur homologue has not been previously identified in V. harveyi.

In addition to regulation of Lux and siderophore, the present data show that LuxO and σ⁵⁴ are involved in the regulation of the rugose colony morphology phenotype. In V. cholerae, the rugose phenotype requires a large gene cluster called vps that is necessary for the production of exopolysaccharide. In V. parahaemolyticus a homologue of the V. harveyi LuxR transcriptional activator protein called OpaR is involved in the switch to the opaque phenotype. We suspect that besides LuxO and σ⁵⁴, genes similar to the vps genes as well as luxR are necessary for the V. harveyi rugose phenotype.

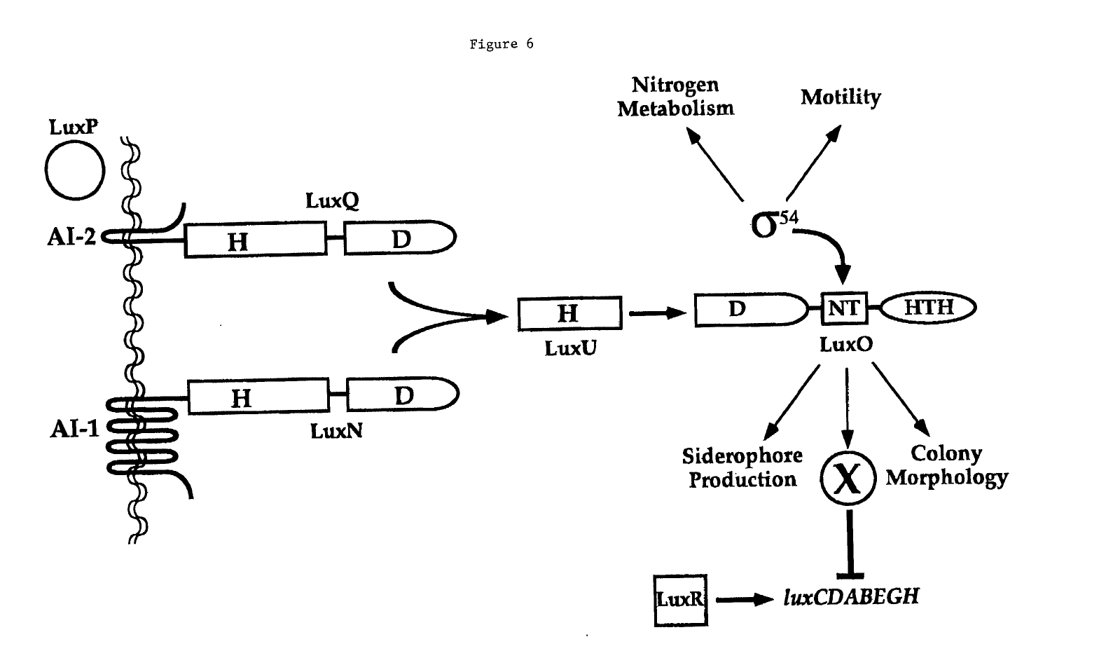

When V. harveyi is at low cell density and low levels of autoinducers are present, we have shown that the hybrid sensors LuxN and LuxQ are kinases. They autophosphorylate at conserved His residues and transfer phosphate to the conserved Asp residues in their receiver domains. Subsequently, phospho-transfer to LuxU occurs, and in the final step, LuxU donates the phosphate to LuxO. Phospho-LuxO is active. Based on the present results, we propose that σ⁵⁴ can interact with phospho-LuxO, and together promote the activation of transcription of some unknown factor (called X in FIG. 6). In this model, the unknown protein X is a negative regulator of luxCDABEGH, so activation of transcription of X results in repression of light production. Additionally, phospho-LuxO and σ⁵⁴ are responsible for activation of genes involved in siderophore production and those required for the switch to the rugose colony morphology.

As the cells grow, the autoinducers AI-1 and AI-2 accumulate and are recognized by their cognate sensors, LuxN for AI-1 and LuxPQ for AI-2. Interaction with the autoinducers causes the sensors LuxN and LuxQ to switch from kinase mode to phosphatase mode. We have shown that the phosphatase activities of the sensors result in the rapid dephosphorylation of LuxO, and this activity is dependent on the phosphorelay protein LuxU. Dephosphorylated LuxO is inactive. We propose that, once dephosphorylated, LuxO cannot activate transcription of X, the proposed negative regulator of Lux, nor can LuxO activate transcription of genes involved in siderophore production and the rugose colony morphology. Decreased transcription of the negative regulator X (and presumably inactivation or degradation of already transcribed X protein) would eliminate repression, and allow transcription of the luxCDABEGH operon and light production at high cell density. However, in this model, and consistent with our results, siderophore production would decrease and V. harveyi would not have the rugose colony morphology at high cell density.

These present data show that the V. harveyi quorum sensing circuit is used to differentially regulate at least three different outputs, light emission, siderophore production and colony morphology. Specifically, the present study shows that the quorum sensing circuit is designed to facilitate both positive and negative regulation of genes in response to changes in cell population density. Differential regulation is accomplished at the level of LuxO, because this is the point where the signal transduction cascade diverges into distinct positively and negatively regulated pathways.

LuxO Contains a σ⁵⁴ Activation Domain.

LuxO is a homologue of the two-component response regulator protein NtrC. NtrC is a member of a growing family of proteins that activate gene transcription in concert with the alternative sigma factor σ⁵⁴. Members of this family of transcriptional activator proteins possess a highly conserved central region that contains nucleotide binding and hydrolysis determinants that are essential for the conversion of closed σ⁵⁴-holoenzyme-promoter complexes into transcriptionally active open complexes. Additionally, oligomerization of these proteins has been shown to be required for activation of transcription. In general, the N-terminal domains of the σ⁵⁴ activator proteins are involved in regulating transcriptional activation in response to environmental cues, often via a two-component response regulator domain. DNA binding helix-turn-helix motifs are located at the C-termini of the majority of these proteins, and this region mediates the binding of the activator proteins to enhancer sequences upstream of the σ⁵⁴ promoter.

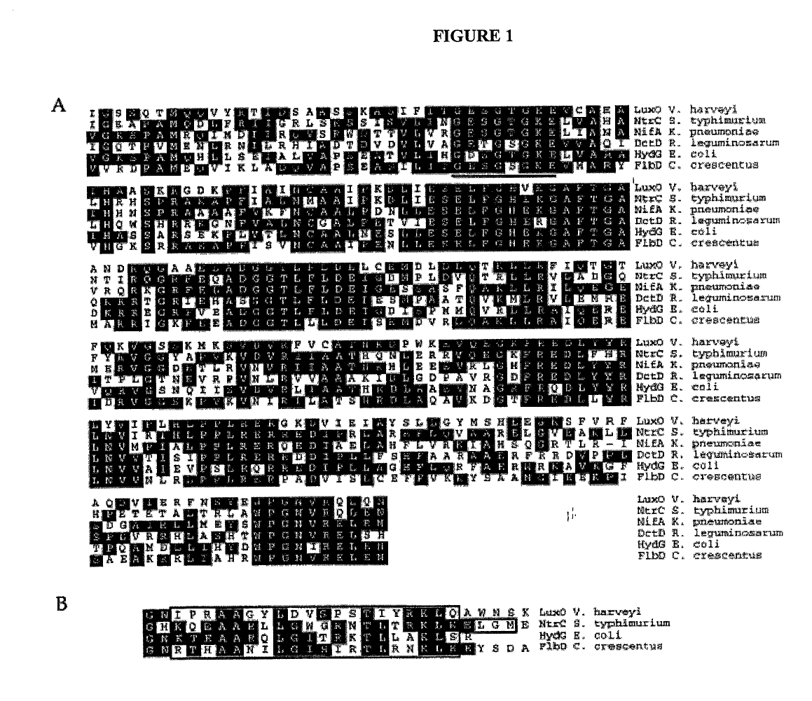

FIG. 1A shows an alignment of the central portion of LuxO to that of five other proteins containing σ⁵⁴ activation domains. The homologous proteins shown in the figure include NtrC of S. typhimurium, NifA of Klebsiella pneumoniae, DctD of Rhizobium leguminosarum, HydG of E. coli, and FlbD of Caulobacter crescentus. In the alignment, amino acids that match the consensus generated for this group of protein sequences are shaded in black. Portions of these sequences (W/FPGNV (SEQ ID NO:4 and ELFGH(V/A/D/E/G) (SEQ ID NO:5) have been used in the design of degenerate primers capable of specifically amplifying σ⁵⁴ activator proteins from the chromosomes of different bacteria. In FIG. 1A, the region that makes up the glycine rich nucleotide binding motif is underlined. The alignment shows that LuxO possesses conserved blocks of sequence that are characteristic of σ⁵⁴ transcriptional activators, including the region that forms the nucleotide binding motif. The high degree of identity between LuxO and the other proteins strongly suggests that LuxO is a σ⁵⁴-dependent transcriptional activator.

FIG. 1B shows an alignment of a region near the C-terminus of LuxO with that of NtrC, HydG and FlbD. The boxed residues delineate the extent of the helix-turn-helix (HTH) DNA binding domains of the various proteins LuxO contains several identical and similar residues in this region, including a pair of alanine residues, which are highly conserved in the HTH domains of various σ⁵⁴ transcriptional activators. When scored using the method of Dodd and Egan (Nucleic Acids Res 18:5019, 1990), which predicts the probability of a sequence forming an HTH domain, the boxed residues in LuxO give a more significant score than the known NtrC HTH domain indicating that this region is highly likely to form an HTH, and to mediate DNA binding by LuxO.

Cloning, Mutagenesis and Analysis of rpoN in V. harveyi

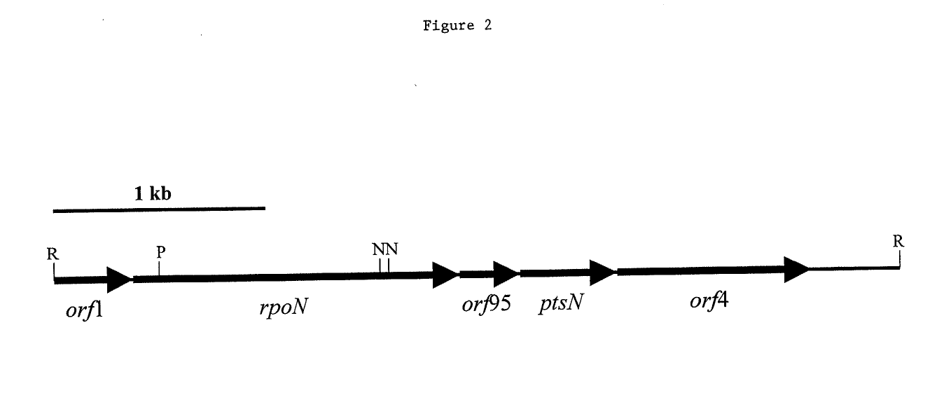

The rpoN gene was PCR amplified from the V. harveyi chromosome using degenerate primers. The PCR product was used to probe a wild type V. harveyi genomic library to obtain cosmids containing the rpoN gene and flanking DNA. Subsequently, a single 4 kb EcoRI fragment containing the rpoN gene was isolated, subcloned and sequenced in its entirety. FIG. 2 shows the genetic organization of the region of the V. harveyi chromosome surrounding the rpoN gene. The region of DNA encompassing rpoN in V. harveyi very closely resembles that surrounding rpoN in V. cholerae and E. coli. The partial ORF upstream of rpoN (orf1) is predicted to encode a protein that is 85% identical to the E. coli YhbG putative ATP binding cassette (ABC) type transporter. The ORFs downstream of rpoN are predicted to encode a putative σ⁵⁴ regulatory protein (83% identical to V. cholerae orf95, Klose and Mekalanos, Mol Microbiol 28:501, 1998), a nitrogen regulatory phosphotransferase component (79% identical to V. cholerae ptsN) and a conserved hypothetical ORF of unknown function (52% identical to E. coli orf4, Jones et al., Microbiol 140:1035, 1994). The rpoN gene of V. harveyi is predicted to encode a protein of 491 amino acids that is highly similar to RpoN proteins from other species including V. alginolyticus (96% identical), V. cholerae (79% identical) and E. coli (60% identical).

A null mutation was constructed in the cloned V. harveyi rpoN gene by introducing a Cm^{r} cassette into the gene using endogenous NsiI sites (see FIG. 2). A V. harveyi rpoN null mutant strain (BNL240) was next constructed by introducing the rpoN::Cm^{r} null allele onto the V. harveyi chromosome at the rpoN locus. In enteric bacteria such as E. coli, S. typhimurium and V. cholerae, σ⁵⁴ (in concert with NtrC) is required for the expression of glutamine synthetase. Specifically, under conditions of nitrogen deprivation, phospho-NtrC oligomerizes and hydrolyzes ATP which provides the energy for the formation of open complexes at the glnA promoter. Therefore, the role of NtrC, together with σ^{54,} is to promote the activation of transcription of glnA when bacteria need nitrogen. Consistent with a similar role for σ⁵⁴ in nitrogen metabolism in V. harveyi, all of our V. harveyi strains containing the rpoN::Cm^{r} allele (Table 4) exhibit growth defects when grown in minimal AB medium, but grow at wild type rates when AB medium is supplemented with L-glutamine. In contrast, LuxO mutants show no requirement for glutamine. These results show that, although LuxO is an NtrC homologue, the role of σ⁵⁴ in nitrogen metabolism is independent of LuxO. Presumably, a true NtrC protein exists in V. harveyi and acts with σ⁵⁴ to regulate nitrogen metabolism.

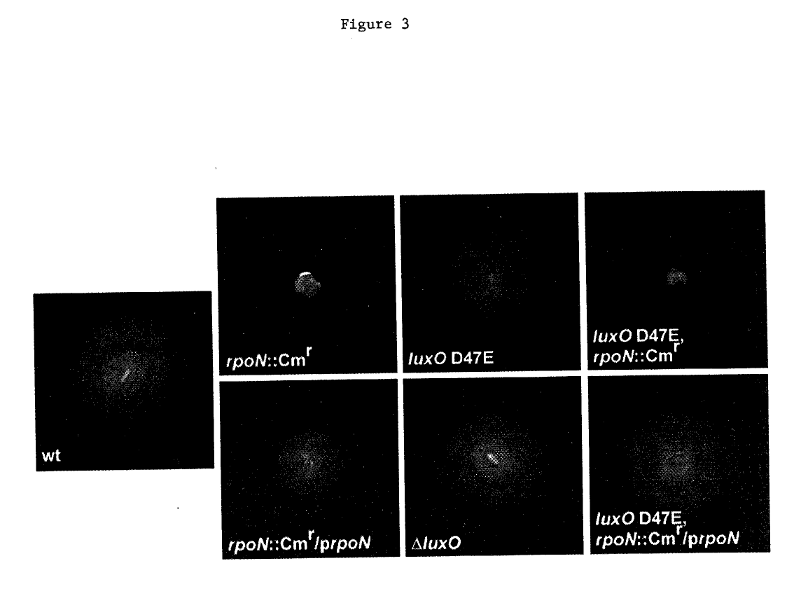

In several species of bacteria including C. crescentus, Pseudomonas putida, V. alginolyticus, V. anguillarum and V. cholerae, σ⁵⁴ is required for transcription of flagellar genes, and rpoN mutants in these species are non-motile. Using soft agar motility plates we tested whether σ⁵⁴ is also required for motility in V. harveyi. The results are shown in FIG. 3. Wild type V. harveyi produces swarm rings in soft agar LM plates. However, the rpoN::Cm^{r} null strain BNL240 is non-motile. In trans expression of wild type rpoN restores motility to strain BNL240. These results show that in V. harveyi, as in other bacteria, σ⁵⁴ is required for motility.

LuxO regulation of motility in V. harveyi was examined using the swarm plate assay system. The motility of a V. harveyi strain carrying a luxO mutation (luxO D47E) that encodes a LuxO protein that is “locked” in a form mimicking activated, phospho-LuxO was asayed. Phospho-LuxO is responsible for repression of the expression of luminescence, so strains carrying activated luxO alleles such as luxO D47E have a dark (Lux⁻) phenotype. FIG. 3 shows that the V. harveyi luxO D47E strain JAF548 forms wild type swarm rings. FIG. 3 also shows that the ΔluxO strain JAF78 forms swarm rings as well as the wild type. Therefore, neither the presence of constitutively active LuxO nor the absence of LuxO impairs motility in V. harveyi. These results indicate that LuxO has no role in regulating motility in V. harveyi. The rpoN::Cm^{r} null mutation eliminated motility in a strain carrying the luxO D47E allele (strain BNL244), and in trans expression of wild type rpoN complemented the motility defect. Therefore, σ⁵⁴ controls motility in V. harveyi, and similar to its role in nitrogen metabolism, σ⁵⁴ regulation of motility is independent of LuxO. Regulation of motility is can involve V. harveyi homologues of other σ⁵⁴-interacting proteins such as FlbD in C. crescentus or FlrA and FlrC in V. cholerae.

σ⁵⁴ is Required for Density Dependent Regulation of Lux Expression in V. harveyi.

LuxO is required for the control of quorum sensing in V harveyi. The following data further indicate that σ⁵⁴ is required for density dependent Lux expression in V. harveyi.

The Lux phenotype of the rpoN::Cm^{r} null strain BNL240 was assayed and compared to that of the wild type strain BB120 and the ΔluxO strain JAF78. The phenotypes of the three strains are shown in FIG. 4. The strains were grown to high cell density and then diluted 1:5000. The light emitted per cell (relative light units or RLU) was measured during the subsequent growth of the cultures. FIG. 4 shows that, at the start of the experiment, the light emitted by the wild type strain is maximal, over 10⁵ RLU (squares). Immediately after dilution, light production by the wild type strain declines over 1000-fold. This decrease in light emission occurs because dilution of the culture at the start of the experiment reduces the concentration of extracellular autoinducers to below the threshold level for detection. However, as the wild type cells grow, they produce autoinducers that accumulate in the environment. The wild type strain BB120 responds to the buildup of autoinducer by inducing light production. In FIG. 4, the response to the autoinducers by the wild type strain can be observed by the rapid, 1000-fold increase in light production. At the end of the experiment, the wild type culture has again attained the pre-dilution level of light production.

The phenotype of the luxO deletion strain JAF78 is different from the wild type (triangles). Strain JAF78 displays maximal constitutive light production at all cell densities, and this phenotype does not depend on the presence of autoinducers (FIG. 4 and Freeman and Bassler, 1999a). The phenotype of the ΔluxO mutant demonstrates that the function of wild type LuxO is to cause repression of the expression of luminescence at low cell densities and low autoinducer concentrations. FIG. 4 shows that the rpoN null mutant V harveyi strain BNL240 has a phenotype identical to that of the ΔluxO strain JAF78, i.e., maximal constitutive luminescence (circles).

LuxO Requires σ⁵⁴ to Regulate Light Production in V. harveyi.

The results in FIG. 4 show that, like LuxO, σ⁵⁴ is required for repression of the expression of luminescence at low cell densities. LuxO contains a σ⁵⁴ interaction domain, indicating that LuxO requires σ⁵⁴ to function in the Lux signaling cascade. To confirm this, the “locked” activated allele of luxO (luxO D47E) was combined with the rpoN null allele and assayed to determine whether the activated LuxO phenotype is dependent on rpoN. The results are presented in Table 1.

TABLE 1
LuxO requires σ⁵⁴ to control the expression of bioluminescence in
V. harveyi.

| V. harveyi strain | Genotype | P_{lac}-rpoN^{a} | % W.T. Lux^{b} |
|---|---|---|---|
| JAF78 | ΔluxO::Cm^{r} | − | 195 ± 9 |
| BNL240 | rpoN::Cm^{r} | − | 215 ± 9 |
| BNL240 | rpoN::Cm^{r} | + | 135 ± 6 |
| JAF548 | luxO D47E | − | .002 |
| BNL244 | luxO D47E, rpoN::Cm^{r} | − | 77 ± 1 |
| BNL244 | luxO D47E, rpoN::Cm^{r} | + | 1.4 ± 2 |
| JAF549 | luxN L166R | − | .004 |
| BNL248 | luxN L166R, rpoN::Cm^{r} | − | 55 ± 2 |
| BNL248 | luxN L166R, rpoN::Cm^{r} | + | 1.1 ± 3 |
| ^{a}The wild type V. harveyi rpoN gene was expressed under control of the lac promoter from plasmid pBNL2090 (Table 4). | | | |
| ^{b}Overnight cultures of V. harveyi were diluted 1:5000 into fresh AB medium (containing Tet for strains carrying pBNL2090) and allowed to grow to high cell density (˜10⁸ CFU ml¹). Subsequently, the light emission and cell density of each culture was measured, and relative light units (RLU) were calculated. The RLU produced by each strain was divided by the RLU produced by the wild type strain, BB120, to determine the % W.T. Lux. Values shown are | | | |
| # the mean ± SEM of three independent experiments. | | | |

In this experiment, different V. harveyi strains were grown to high cell densities and then the light produced per cell was measured. The amount of light emitted by each strain was compared to that produced by the wild type V. harveyi strain BB120. The results for each strain are presented as the percentage of the light produced by the wild type. Table 1 shows that both the ΔluxO strain JAF78 and the rpoN::Cm^{r} null strain BNL240 produce slightly higher levels of light than the wild type strain (195% and 215% respectively). In contrast, V. harveyi strain JAF548 (luxO D47E) emits 50,000-fold less light than wild type V harveyi (0.002%). However, when rpoN was disrupted in the presence of the luxO D47E mutation (strain BNL244) light production increases to nearly the wild type level (77%). This result shows that the luxO D47E phenotype is dependent on rpoN. Table 1 furtheer shows that in trans expression of the wild type rpoN ORF under the control of the lac promoter in BNL244 partially complements the rpoN defect. Specifically, the presence of wild type rpoN causes a reduction in light production from 77% to 1% of the wild type level. The data indicate that the phenotype observed for the rpoN::Cm^{r} strains is due specifically to a defect in rpoN and not to the inactivation of any gene located downstream of rpoN.

Response regulators containing mutations equivalent to the LuxO D47E mutation are not phosphorylated; they merely mimic the phosphorylated form. The present invention provides “locked” luxN allele (luxN L166R) that encodes a LuxN protein with constitutive kinase activity was combined with the rpoN::Cm^{r} null mutation to further shoe that phospho-LuxO cannot act in the absence of σ⁵⁴. The LuxN L166R protein does not recognize AI-1, and therefore it never switches from the kinase mode to the phosphatase mode. In strains carrying the luxN L166R mutation, LuxO is always phosphorylated, and this results in constitutive repression of Lux and a dark (Lux⁻) phenotype.

Table 1 shows that, like the luxO D47E strain JAF548, strain JAF549 (luxN L1 66R) produces almost no light (0.004% or 25,000-fold less than the wild type level). Similar to the results for the luxO D47E strain, the rpoN::Cm^{r} null mutation is epistatic to the luxN L166R mutation. Introduction of the rpoN::Cm^{r} null mutation onto the chromosome of JAF549 (strain BNL248), increases light production from 0.004% to 55% of the wild type level. Again, in trans introduction of wild type rpoN results in partial complementation of the rpoN::Cm^{r} defect, and light emission is repressed to 1% of the wild type level. The results presented in Table 1 show that phospho-LuxO requires σ⁵⁴ to function. Therefore, the involvement of σ⁵⁴ in regulation of Lux quorum sensing is via LuxO and not some other, unidentified pathway.

LuxO and σ⁵⁴ Do Not Regulate the Transcription of LuxO.

A plasmid containing a luxO-lacZ transcriptional reporter fusion (pBNL2078) was constructed and its expression measured in the wild type V. harveyi strain BB120, in strain JAF548 (luxO D47E) and in strain BNL240 (rpoN::Cm^{r}) to show that the transcription of luxO does not require rpoN, nor does activated LuxO and σ⁵⁴ regulate the expression of luxO.

In the experiment presented in Table 2, each strain was grown to high cell density and β-galactosidase activity was measured. The results are shown in Miller units. Each result is the average of three independent experiments. Table 2 shows that, in the wild type V. harveyi strain BB120, at high cell density, the level of β-galactosidase activity is 969 Miller units. The presence of constitutively active LuxO (strain JAF548) does not affect the expression of the luxO-lacZ reporter (845 Miller units). Likewise, the absence of rpoN (strain BNL240) does not dramatically affect expression of luxO (763 Miller units). Taken together, these results indicate that neither LuxO nor σ⁵⁴ is involved in regulation of the transcription of luxO.

TABLE 2
LuxO and σ⁵⁴ do not regulate the transcription of luxO

| V. harveyi strain^{a} | Genotype | luxO-lacZ activity (Miller units)^{b} |
|---|---|---|
| BB120 | wild type | 969 ± 97 |
| JAF548 | luxO D47E | 845 ± 91 |
| BNL240 | rpoN::Cm^{r} | 763 ± 82 |

^{a}Each strain contains the luxO-lacZ transcriptional reporter fusion present on plasmid pBNL2078 (Table 4).
^{b}Values shown are the mean ± SEM of three independent experiments.

σ⁵⁴ and LuxO Regulate Additional Phenotypes in V. harveyi.

The present study demonstrates that LuxO, in conjunction with, σ⁵⁴ regulates the density dependent expression of luminescence. The study further indicates that targets other than Lux are under LuxO-σ⁵⁴ control. For example, the concentration of iron in a bacterial growth medium affects density dependent Lux expression. Genes involved in iron acquisition may control by quorum sensing in V. harveyi. In the present study, mutations in luxO and/or rpoN were tested to determine if they affected siderophore production in V. harveyi. The Schwyn and Neilands chromazurol S assay was used to measure siderophore released by different V. harveyi strains. The S assay quantitatively measures siderophore by optically assessing the color change that chromazurol S undergoes when iron is chelated from it by siderophore present in spent culture fluids. The results are presented in Table 3.

TABLE 3
Siderophore production in V. harveyi is regulated by LuxO and σ⁵⁴

| V. harveyi | | | |
|---|---|---|---|
| strain | Genotype | P_{lac}-rpoN^{a} | Siderophore units^{b} |
| BB120 | wildtype | − | 8 ± 3 |
| JAF78 | ΔluxO::Cm^{r} | − | 7 ± 4 |
| JAF548 | luxO D47E | − | 50 ± 5 |
| BNL240 | rpoN::Cm^{r} | − | 3 ± 3 |
| BNL240 | rpoN::Cm^{r} | + | 6 ± 3 |
| BNL244 | luxO D47E, rpoN::Cm^{r} | − | 4 ± 1 |
| BNL244 | luxO D47E, rpoN::Cm^{r} | + | 25 ± 3 |

^{a}The wild type V. harveyi rpoN gene was expressed under control of the lac promoter from plasmid pBNL2090 (Table 4).
^{b}Siderophore production was measured using the chromazurol S assay (Schwyn and Neilands, 1987). Siderophore units were calculated according to the method of Payne (1994), and normalized for cell number using the formula: 100 X [(OD₆₃₀ (media control) - OD₆₃₀ (spent culture fluid))/OD₆₀₀ (cell culture)]. Values shown are the mean ± SEM of three independent experiments.

The wild type strain BB120, the ΔluxO strain JAF78, and the rpoN::Cm^{r} null strain BNL240 all produce similar amounts of siderophore (3 to 8 units) when grown in AB minimal medium. In contrast, the presence of activated LuxO D47E in JAF548 increases siderophore production to 50 units. This result indicates that phospho-LuxO activates siderophore production. Disruption of rpoN in the luxO D47E background (strain BNL244) reduces siderophore production to wild type levels (4 units), indicating that similar to what was shown above for Lux regulation, phospho-LuxO can only control siderophore production when wild type σ⁵⁴ is present. In trans introduction of wild type rpoN into the luxO D47E, rpoN::Cm^{r} strain complements the defect. In this case, siderophore production increased to 25 units, approaching that of the luxO D47E strain. The results of this assay demonstrate that the activated form of LuxO has a role in regulation of siderophore production in V. harveyi, and σ⁵⁴ is required for this effect.

In addition to the siderophore production phenotype, the present study shows that V harveyi mutants possessing a constitutively activated LuxO (i.e., LuxO D47E or LuxN L166R) also consistently exhibit an altered colony morphology that is similar to the rugose colony morphology described for V cholerae and the opaque colony morphology described for Vibrio parahaemolyticus. The rugose variants of V. cholerae have been shown to form pellicles in liquid culture, and to produce an exopolysaccharide matrix that mediates resistance to chlorine and enhances biofilm formation.

FIG. 5 shows the colony morphologies of various V. harveyi strains. Colonies of wild type V. harveyi and the rpoN::Cm^{r} null strain are smooth and glassy in appearance, while colonies of the luxO D47E strain are wrinkled and opaque. The figure shows that the colony morphology phenotype caused by the activated LuxO D47E protein is dependent upon the presence of wild type rpoN because strain BNL244 (luxO D47E, rpoN::Cm^{r}) has the wild type smooth colony morphology. Similar to that observed for rugose strains of V. cholerae, the V. harveyi luxO D47E mutant forms a pellicle when grown in liquid culture. Pellicle formation is also dependent on wild type rpoN. Identical results to those shown in FIG. 5 were obtained when the “locked” luxN L166R strain JAF549 was used in place of the luxO D47E strain JAF548. The fact that a single amino acid change in LuxO or LuxN can affect three different phenotypes (Lux, siderophore production and colony morphology), and that a null mutation in rpoN is epistatic to the LuxO and LuxN mutations with respect to all three phenotypes indicates that LuxO and σ⁵⁴ are involved in the regulation of multiple target genes.

Experimental Procedures

Bacteria Strains and Media.

V. harveyi strains used in the present study along with their relevant properties are listed in Table 4. V. harveyi strains were grown at 30° C. in Heart Infusion (HI) medium containing (per liter): 20 g NaCl, 25 g Heart Infusion Broth (Difco Laboratories) prior to preparation of chromosomal DNA. Density dependent bioluminescence assays, siderophore production assays and β-galactosidase assays were performed on V. harveyi strains that had been grown in autoinducer bioassay (AB) medium (Greenberg et al., Arch Microbiol 120:87, 1979). Cell densities were determined by diluting and plating V. harveyi onto solid LM (L-Marine) medium. LM contains (per liter): 20 g NaCl, 10 g Bacto-Tryptone (Difco Laboratories), 5 g Bacto-Yeast Extract (Difco Laboratories). V. harveyi rpoN::Cm^{r} strains were supplemented with 1 mM L-Glutamine (Sigma) during growth in LM and AB. E. coli strain JM109 [supE Δ(lac-proAB) hsdR17 recaA1 F′ traD36 proAB⁺ lacI^{q} lacZΔM15] was used for propagation of cloned V. harveyi genomic DNA and for DNA preparation for sequencing. E. coli CC118 [araD139 Δara leu76a7 ΔlacX74 ΔphoA20 galE galK thi rpsE rpoB argE (Am) recA1] containing the plasmids pRK2013 (tra) or pPH1JI (tra, mob) was used in conjugations with V. harveyi to construct allelic replacements (Bassler et al., Mol Microbiol 9:773, 1993). E. coli strains were grown in LB (per L: 10 g bacto-tryptone, 5 g bacto-yeast extract and 10 g NaCl) medium at 37° C. with antibiotics at the concentrations specified below. When solid medium was required, 15 g of agar was added per liter prior to sterilization, except for HI-medium to which 20 g of agar was added. Antibiotics (Sigma) were added to media at the following concentrations: (mg/L) ampicillin (Amp), 100; kanamycin (Kan), 100; tetracycline (Tet), 10; gentamycin (Gent), 100 and chloramphenicol (Cm), 10.

TABLE 4
V. harveyi strains and plasmids used in this study

| Strain/Plasmid | Relevant Genotype or Feature |
|---|---|
| BB120 | wild type |
| JAF78 | ΔluxO-Cm^{r} |
| JAF548 | luxO D47E linked to Kn^{r} |
| JAF549 | luxN L166R linked to Kn^{r} |
| BNL240 | rpoN::Cm^{r} |
| BNL244 | rpoN::Cm^{r}, luxO D47E linked to Kn^{r} |
| BNL248 | rpoN::Cm^{r}, luxN L166R linked to Kn^{r} |
| p34S-Cm2 | Cm^{r} Cassette |
| pACYC184 | Medium copy cloning vector, Tet^{r}, Cm^{r} |
| pLAFR2 | Broad Host Range; mob, Tet^{r} |
| pPH1JI | Broad Host Range; tra, mob |
| pRK415 | Broad Host Range, mob, P_{lac}, Tet^{r} |
| pRK2013 | Broad Host Range; tra |
| pUC18 | High copy cloning vector, Amp^{r} |
| pBNL148 | pLAFR2 with rpoN on ˜25 kb genomic fragment |
| pBNL162 | pACYC184 with 4 kb rpoN subclone |
| pBNL2018 | pLAFR2 with rpoN::Cm^{r} allele |
| pBNL2022 | pACYC184 with rpoN ORF |
| pBNL2078 | pLAFR2 with luxO::Tn5lac (Tn5-B20) |
| pBNL2090 | pRK415 with P_{lac}-rpoN ORF |

Assays.

V. harveyi density dependent and high cell density bioluminescence assays were performed as described in Bassler et al., (Mol Microbiol 9:773, 1993) and Freeman and Bassler (Cell-Cell Signaling in Bacteria, Washington, D.C.: American Society for Microbiology Press, pp. 259-273, 1999), respectively. Siderophore production was measured using the liquid chromazurol S assay described in Schwynn and Neilands (Anal Biochem 160:47, 1987), and siderophore units were quantitated according to the method of Payne (Methods Enzymol 235:329, 1994), but values were normalized for cell density. We applied the following formula to calculate the normalized siderophore units: 100×[(OD₆₃₀ (media control)−OD₆₃₀ (spent culture fluid))/OD₆₀₀ (cell culture)]. β-galactosidase assays were performed according to the method of Miller (1992). V. harveyi strains were assayed for motility by inoculating strains using a sterile needle into soft agar LM plates (3 g agar/L). The motility plates were subsequently incubated upright at 300C for 14 hr, after which photographs were taken.

DNA Isolation, Manipulation and Analysis.

DNA isolation, restriction analysis and transformations of E. coli were performed as described in Sambrook et al. Restriction enzymes and T4 DNA ligase (New England Biolabs); Taq DNA polymerase and Calf Alkaline Phosphatase (Boehringer-Mannheim); Pfu DNA polymerase (Stratagene) were used according to manufacturer's specifications. Sequencing grade DNA was prepared with the Qiagen Miniprep kit, and all primers were synthesized by Midland Certified Reagent Company (Midland, Tex.). DNA sequencing was performed by the Princeton University DNA Synthesis/Sequencing Facility using an automated dideoxy chain termination method. Extraction of DNA from agarose gels was performed with the Qiagen Qiaquick Gel Extraction kit. Southern blots and V. harveyi chromosomal DNA preparations were performed according to the method of Martin et al. (1989). Radiolabeled DNA probes used in Southern blots were generated using [α³²P]dATP (NEN Life Sciences) and the Multiprime DNA labelling kit (Amersham). Amplification of V. harveyi genes directly from the chromosome was accomplished using the polymerase chain reaction (PCR). When necessary, PCR products were purified using the Qiaquick PCR Purification kit (Qiagen).

Identification, Cloning and Sequencing of rpoN from V. harveyi.

In order to amplify the V. harveyi rpoN gene from the chromosome, degenerate oligonucleotide primers were constructed based on the rpoN sequences of different Vibrio species. The sequences of the upstream and downstream primers used to amplify the V. harveyi rpoN gene are as follows:

(SEQ ID NO:6) 5′-GGYCAACARTTAGCSATGAC-3′ and

(SEQ ID NO:7) 5′-CATSGCYTCYTCWCCATACTC-3′

The product of the PCR reaction was purified and used to probe a V. harveyi genomic DNA cosmid library. The preparation of the V. harveyi genomic library and the methods used to probe this library have been described previously (Showalter et al., J Bacteriol 172:2946, 1990). Cosmid DNA from the library that hybridized to the V. harveyi rpoN PCR product was analyzed by restriction analysis and Southern blotting. All of the clones identified contained overlapping fragments of V. harveyi genomic DNA. One clone, pBNL148, was used for further analysis. A single 4 kb V. harveyi EcoRI genomic fragment from pBNL148 was shown to hybridize to the labeled rpoN PCR product by Southern blot. This fragment was subsequently subcloned into the vector pACYC184 (New England Biolabs), and the resulting plasmid, pBNL162, was used for sequencing of the V. harveyi DNA. The sequence data were analyzed using the BLAST NCBI website. Alignments shown in FIG. 1 were generated using the Clustal multiple sequence alignment function of the MegAlign program (DNAstar). The V. harveyi rpoN sequence has been deposited in Genbank and has the Accession number AF227983.

Construction of a V. harveyi rpoN::Cm^{r} Null Mutant.

The plasmid pBNL162, containing the V. harveyi rpoN gene on a 4 kb EcoRI fragment, was used for the construction of a null mutation in the rpoN gene as follows. Plasmid pBNL162 was digested with the enzyme NsiI which acts at two endogenous sites within the rpoN gene (see FIG. 2). The Cm^{r} cassette contained on p34S-Cm2 was isolated by restriction digestion of p34S-Cm2 with PstI. This procedure generates compatible cohesive ends with NsiI. The Cm^{r} cassette was next ligated into the NsiI digested pBNL162. The resulting construction containing rpoN::Cm^{r} is called pBNL172. The EcoRI fragment containing the rpoN::Cm^{r} allele and flanking DNA regions from pBNL172 was subsequently cloned into the broad host range cosmid pLAFR2, resulting in pBNL2018. This construction was used for introduction of the rpoN::Cm^{r} allele onto the chromosome of several V. harveyi strains (Table 4). The presence of the rpoN::Cm^{r} allele at the proper location in the V. harveyi chromosome was confirmed using PCR with primers specific for the rpoN ORF as well as with Southern blot using the rpoN ORF as a probe.

Construction of a Vector Carrying RpoN for in Trans Expression in V. harveyi.

The wild type V. harveyi rpoN gene was cloned into the broad host range vector pRK415 for in trans expression in V. harveyi. To accomplish this, the V. harveyi rpoN gene contained on pBNL162 was amplified by PCR using the upstream and downstream primers:

(SEQ ID NO:8) 5′-GGAACGGTA GAATTCTGAGCATTAC-3′ and

(SEQ ID NO:9) 5′-CCTTTT GAATTCGTGCCTAAAGTAGGCG-3′

These primers contain EcoRI restriction sites (underlined). After amplification, the PCR product was digested with EcoRI followed by ligation into EcoRI digested pACYC184 resulting in plasmid pBNL2022. The rpoN containing fragment in pBNL2022 was sequenced to ensure that no mutations had been introduced during PCR amplification. To construct an rpoN expression construct for use in V. harveyi, plasmid pBNL2022 was digested with EcoRI, and the rpoN ORF was subsequently cloned into EcoRI digested pRK415. This construction is called pBNL2090.

Construction of a luxO-lacZ Transcriptional Reporter Fusion for Expression in V. harveyi.

The luxO gene contained on a V. harveyi EcoRI genomic fragment has been subcloned into the broad host range cosmid pLAFR2. This construction was mutagenized in E. coli with λ::Tn5-B20 to obtain luxO-lacZ transcriptional fusions. The method used for transposon mutagenesis was described in Bassler et al. (Mol Microbiol., 9:773, 1993). One such luxO-lacZ fusion plasmid, called pBNL2078, was transferred into several V. harveyi recipient strains by conjugation. The level of luxO-lacZ transcriptional activity was examined using assays to measure β-galactosidase production.

SEQ ID NO:1

| | |
|---|---|
| 1 | agctcacggt ctttcattgc catacgggaa ttccatatac agcacatacg caccagtgcg |
| 61 | ggtatggcac tatcaggtgg tgaacgccgc cgtgtagaaa ttgctcgtgc attggcagca |
| 121 | aaccctcagt tcattttgtt ggatgaaccg ttcgcgggtg ttgacccaat ttcggttaac |
| 181 | gacatcaaaa aaatcatcga acacttgcgc gatcgcggcc ttggcgtgtt aatcacagac |
| 241 | cataacgtac gcgaaacctt ggacgtttgt gaaaaagcct atatcgtaag ccaaggacac |
| 301 | ctcatcgcat cgggaactcc ggatgaagtt ctcaataacg agcaagtgaa acaagtttat |
| 361 | ctcggcgaac aattccgtct atgattacat taggaacggt aagattctga gcattacaag |
| 421 | gtaagtaaca ctgaatgaaa ccttcattac aactcaagct aggtcaacag ttagccatga |
| 481 | cgccacagct gcagcaagcg attcgtttgt tgcaattgtc gacgctcgat cttcaacaag |
| 541 | aaatccaaga agcgttggac tccaacccgc tactggaagt tgaagaaggc cacgatgagc |
| 601 | ctcaagcaaa tggtgaagac aaatcagcgt ctgaatctgc tgataaaagt gcgaacgaag |
| 661 | ctaacgatgc ctcagaaccc gaccttccag atagctcaga cgtgattgaa aaatctgaaa |
| 721 | tcagctctga gctagaaatt gataccactt gggatgacgt atatagcgca aacacgggca |
| 781 | gcacaggcct agcgctggat gatgacatgc ccgtctacca aggtgagacc actgaatctt |
| 841 | tgcatgatta ccttatgtgg cagttagact taacgccttt cagtgaaacc gaccgcacca |
| 901 | tcgccctcgc gattatcgat gcggtcgacg actacggcta cttaacccta tcccctgaag |
| 961 | aaattcacga gagcttcgac aacgaagaag tggaattgga tgaagtagaa gcggtacgta |
| 1021 | agcgtattca gcaatttgac ccgctcggtg tagcctctcg caatctgcaa gaatgcctac |
| 1081 | tgctacaact ggcaactttc cctgaagaca cgccgtggct tgctgaggcg aaaatggtgt |
| 1141 | tgagcgatca catcgaccac cttggcaatc gtgactacaa gctggtcatc aaagaggcta |
| 1201 | agcttaaaga agcggacttg cgtgaagtat tgaagttgat tcaacaactt gacccacgtc |
| 1261 | caggtagtcg tatcacaccc gatgacactg aatacgtcat tccggatgtg tccgtattta |
| 1321 | aagatcatgg taagtggacc gtctccataa accctgacag cattccgaaa ctaaaagtaa |
| 1381 | atcaacaata tgcgcaacta ggcaaaggca acagtgcgga tagccagtac attcgcagca |
| 1441 | atttgcaaga ggcaaaatgg ctgattaaga gcctagaaag cagaaacgag acgcttctca |
| 1501 | aagttgcaag atgtattgtt gaacatcaac aagatttctt cgagtatggt gaagaagcca |
| 1561 | tgaaaccaat ggtgctaaac gacgtagcat tggatgtgga catgcatgaa tcgacaattt |
| 1621 | ctcgtgtaac aacacagaag tttatgcata ccccacgtgg catttttgaa ttgaagtact |
| 1681 | tcttctctag ccatgttagt acagacaatg gtggagagtg ttcgtccaca gcaattcgcg |
| 1741 | cactcatcaa aaagttggtc gcagcggaga ataccgctaa gccactgagt gatagcaaaa |
| 1801 | ttgctgctct tctggctgac caggggattc aagtcgcgag acggacgata gcaaaatatc |
| 1861 | gtgaatcctt gggtattgcc ccttcgagtc agcgtaaacg cctactttag gcaccaattg |
| 1921 | aaaaggaaag tctatgcaaa tcaatattca aggccatcac gttgatctta ccgattcaat |
| 1981 | gcaagaatat gttgactcta agtttcaaaa gctcgagcgg ttcttcgacc acatcaatca |
| 2041 | agtccatgtc gtattaaaag ttgaaaaact taaccaaata gccgaagcta cgctccacat |
| 2101 | caatcaaggc gaaatccacg cgtcatcgaa cgacgaaagt atgtatgcag caattgattc |
| 2161 | gctggtggat aaattagttc gtcaacttaa caagcacaaa gaaaaactaa acagtcatta |
| 2221 | atcatgcaat tgagcgaaat actgtcactg gactgcacca aaagtgcggt ccattgtaca |
| 2281 | agtaagaaac gtgccctcga aatgatcagc caaattgtcg ctgaaaacac gggccaagat |
| 2341 | tctacagaac tgtttgagtg tatgctcagc agagaaaaaa tgggtagtac tggtatcggc |
| 2401 | aacggtattg ctatccctca cgcaagaatg caatcaagcg acaaagccat cgcagtgtta |
| 2461 | cttcagtgtg acgaagcaat tgaatttgac gctatcgaca accgacctgt cgaccttctt |
| 2521 | tttgctctcc ttgtacctga agaacagtgc aaagagcacc tcaaaacact atcctctatg |
| 2581 | gcagagcgtc taagtgacaa gcaagtgctt aaaagcttac gtaacgctca gagcgatgaa |
| 2641 | gagctctacg acattatgat tcataagtaa tcaggacgat caccatgcga ttaatcgttg |
| 2701 | ttagcgggca ctctggtgcc gggaaaagtg ttgccctgcg cgtacttgag gacttaggtt |
| 2761 | actactgcgt agacaaccta ccggtaaact tacttgacgc gtttgttcag tcagtctctg |
| 2821 | agagcaaaca aaatgtcgca gtaagcatcg atattcgaaa tatccctaag aagctcaaag |
| 2881 | aactgaatac cacgctagag aagctaaagg ctgaactgga tgtgacagta ctgttcttag |
| 2941 | acgcgaataa agaaacgctt ctcacccgct acagcgaaac acgtcggatt catccgctat |
| 3001 | cacttgacag tcaatcatta tcacttgatc aggcgattga gcttgaacaa gagatcttaa |
| 3061 | tgcctctgaa agcacacgca gacttagttc tgaacagtag cggtcaatct ctgcatgatc |
| 3121 | tcagtgaaac cgtacgtatg cgtgtggaag gccgagaacg caaagactta gtcatggtgt |
| 3181 | ttgagtcgtt tggtttcaaa tacggtttac catcagatgc cgattacgtg tttgatgtgc |
| 3241 | gtttcttgcc aaacccacac tgggagccag cactgcgccc tctcactggt ttagatggcc |
| 3301 | cgatcggcgc cttcttagag caacaccagt cggtacttga tctgaaatac caaattgaaa |
| 3361 | gctttattga gacttggtta ccactattag agaaaaacaa ccgtagttac ctgaccgttg |
| 3421 | cgattggttg tactggtggt aaacaccgct cggtttatct tactcaaaaa attggtgagt |
| 3481 | tctttgcgga caaaggacac caagtacaaa ttcgccacac ttcattggaa aagaacgtta |
| 3541 | aggaataacg gtggaattaa gtcgtaaagt actgatccaa aaccgactag gcttgcacgc |
| 3601 | tcgtgcggca gttaaactgg tagaactagc acaaagcttc gacgcggtga ttaccatcga |
| 3661 | caacgaagaa gacaaaaccg cgaccgcaga cagcgtcatg ggattgctga tgctggaatc |
| 3721 | agcccaagga caatacgtga ccatccacgc cactggcgat caatctgagc aagctcttga |
| 3781 | tgcggtttgc catttgatcg aagataagtt tgacgaaggc gagtgattca ctcgcttttt |
| 3841 | tattatctct agccagatat cccacataag tttcacctcc tgcttaaatt ccgacaaata |
| 3901 | attttgtcga ctttcataag ttgttattaa aaggtgccta gaattaagtt attattcaaa |
| 3961 | gcattgtaaa tatcaggaat tgggaggaat gaatggcaga gca |

SEQ ID NO:2 (435-1910)

MKPSLQLKLG QQLANTPQLQ QAIRLLQLST LDLQQEIQEA LDSNPLLEVE EGHDEPQANG
EDKSASESAD KSANEANDAS EPDLPDSSDV IEKSEISSEL EIDTTWDDVY SANTGSTGLA
LDDDMPVYQG ETTESLHDYL MWQLDLTPFS ETDRTIALAI IDAVDDYGYL TLSPEEIHES
FDNEEVELDE VEAVRKRIQQ FDPLGVASRN LQECLLLQLA TFPEDTPWLA EAKMVLSDHI
DHLGNRDYKL VIKEAKLKEA DLREVLKLIQ QLDPRPGSRI TPDDTEYVIP DVSVFKDHGK
WTVSINPDSI PKLKVNQQYA QLGKGNSADS QYIRSNLQEA KWLIKSLESR NETLLKVARC
IVEHQQDFFE YGEEAMKPMV LNDVALDVDM HESTISRVTT QKFMHTPRGI FELKYFFSSH
VSTDNGGECS STAIRALIKK LVAAENTAKP LSDSKIAALL ADQGIQVARR TIAKYRESLG
IAPSSQRKRLL
SEQ ID NO:3

| | |
|---|---|
| 435 | atgaaa ccttcattac aactcaagct aggtcaacag ttagccatga |
| 481 | cgccacagct gcagcaagcg attcgtttgt tgcaattgtc gacgctcgat cttcaacaag |
| 541 | aaatccaaga agcgttggac tccaacccgc tactggaagt tgaagaaggc cacgatgagc |
| 601 | ctcaagcaaa tggtgaagac aaatcagcgt ctgaatctgc tgataaaagt gcgaacgaag |
| 661 | ctaacgatgc ctcagaaccc gaccttccag atagctcaga cgtgattgaa aaatctgaaa |
| 721 | tcagctctga gctagaaatt gataccactt gggatgacgt atatagcgca aacacgggca |
| 781 | gcacaggcct agcgctggat gatgacatgc ccgtctacca aggtgagacc actgaatctt |
| 841 | tgcatgatta ccttatgtgg cagttagact taacgccttt cagtgaaacc gaccgcacca |
| 901 | tcgccctcgc |

A number of embodiments of the invention have been described. Nevertheless, it will be understood that various modifications may be made without departing from the spirit and scope of the invention. Accordingly, other embodiments are within the scope of the following claims.

What is claimed is:

1. An isolated σ⁵⁴ polypeptide comprising an amino acid sequence set forth in SEQ ID NO:2, and conservative variants thereof.

2. A polynucleotide selected from the group consisting of:
(a) SEQ ID NO:1;
(b) SEQ ID NO:1, wherein T is U;
(c) nucleic acid sequences complementary to (a) or (b); and
(d) fragments of (a), (b), or (c) that are at least 15 nucleotide bases in length and that hybridize to DNA under highly stringent conditions including 0.1× SSC at about 68° C. for 15 minutes which encodes the polypeptide set forth in SEQ ID NO:2.

3. An isolated polypeptide comprising an amino acid sequence encoded by a nucleic acid sequence which hybridizes to the nucleic acid sequence set forth in SEQ ID NO:1 under highly stringent conditions, wherein the highly stringent conditions include 0.1× SSC at about 68° C. for 15 minutes, and wherein the polypeptide is a transcription factor.

4. The polypeptide of claim 3, wherein the polypeptide binds to a LuxO protein.

5. An isolated polypeptide that binds to an antibody generated against the polypeptide of SEQ ID NO:2, or fragments and conservative variants thereof, wherein the polypeptide is a transcription factor.

6. A vector containing the polynucleotide of claim 2.

7. A host cell containing the vector of claim 6.

8. An anti-polypeptide antibody that binds to the polypeptide of claim 3.

9. A method for regulating the activity of a σ⁵⁴ polypeptide comprising contacting σ⁵⁴ with a LuxO polypeptide.

10. The method of claim 9, wherein the contacting is in vivo.

11. The method of claim 9, wherein the contacting is in vitro.

12. The method of claim 9, wherein the σ⁵⁴ polypeptide is from V. harveyi.

13. A method for identifying a compound that regulates the binding of a LuxO polypeptide to a σ⁵⁴ polypeptide comprising:
(a) contacting a σ⁵⁴ polypeptide with a LuxO polypeptide under conditions and for such time as to allow binding of the σ⁵⁴ polypeptide to the LuxO polypeptide;
(b) contacting the σ⁵⁴ polypeptide or LuxO polypeptide of (a) with the compound prior to, simultaneously with, or after binding of the σ⁵⁴ polypeptide to the LuxO polypeptide; and
(c) measuring the binding of the σ⁵⁴ polypeptide to the LuxO polypeptide in the presence of the compound and comparing it to the binding of the LuxO polypeptide with the σ⁵⁴ polypeptide in the absence of the compound,
wherein, a change in the binding of a LuxO polypeptide to a σ⁵⁴ polypeptide in the presence of the compound is indicative of a compound that regulates LuxO-σ⁵⁴ binding.

14. The method of claim 13, further comprising manufacturing the compound so identified.

15. The method of claim 13, further comprising formulating the compound so identified with a pharmaceutically acceptable carrier.

16. The method of claim 13, wherein the contacting is in vivo.

17. The method of claim 13, wherein the contacting is in vitro.

18. The method of claim 13, wherein the modulation is by inhibition of LuxO-σ⁵⁴ binding.

19. The method of claim 13, wherein the compound is a polypeptide.

20. The method of claim 13, wherein the compound is a small molecule.

21. The method of claim 13, wherein the compound is a LuxO analog.

22. The method of claim 13, wherein the compound is a σ⁵⁴ analog.

23. The method of claim 13, wherein the compound is a nucleic acid.

24. A pharmaceutical composition comprising a compound identified by the method of claim 13 in a pharmaceutically acceptable carrier.

25. The pharmaceutical composition of claim 24 in a controlled release formulation.

26. The pharmaceutical composition of claim 25 in a liposomal form.

27. The pharmaceutical composition of claim 24 in a lyophilized form.

28. The pharmaceutical composition of claim 23 in a unit dosage form.

29. A method for identifying a compound that inhibits LuxO-σ⁵⁴ binding comprising:
(a) contacting a mixture comprising LuxO and σ⁵⁴ with the compound under conditions and for such time as to allow LuxO-σ⁵⁴ binding;
(b) contacting (a) with a bacterial cell, or extract thereof, comprising biosynthetic pathways which will produce a detectable amount of light in response to LuxO-σ⁵⁴ binding; and
(c) measuring the effect of the compound on light production,
wherein decreased light production in the presence of the compound, compared to light production in the absence of the compound, identifies the compound as a compound that inhibits LuxO-σ⁵⁴ binding.

30. The method of claim 29, wherein the compound is a competitive inhibitor.

31. The method of claim 29, wherein the compound is a suicide inhibitor.

32. A method for identifying a compound that regulates the activity of a LuxO-σ⁵⁴ complex, comprising:
(a) contacting a LuxO-σ⁵⁴ complex with the compound; and
(b) measuring the activity of the complex in the presence of the compound and comparing the activity of the complex obtained in the presence of the compound to the activity of the complex obtained in the absence of the compound;
wherein a change in the activity of the LuxO-σ⁵⁴ complex in the presence of the compound is indicative of a compound that regulates LuxO-σ⁵⁴ complex activity.

33. A method for regulating expression of a virulence factor in a bacterial cell comprising contacting a bacterial cell capable of producing the virulence factor with a compound identified by the method of claim 13, claim 29 or claim 32.

34. The method of claim 33, wherein the regulating is inhibition of expression of the virulence factor.

35. The method of claim 33, wherein the virulence factor is a siderophore.

36. The method of claim 33, wherein the virulence factor is selected from the group consisting of Accessory cholera enterotoxin, Adenylate cyclase toxin, Adhesin, Aerolysin toxin, aggregation substance, Agr A, B, C, D, SigB etc, Alkaline protease, Alpha toxin, Alpha-haemolysin, Alveolysin, Anthrax toxin, APX toxin, Beta toxin, Botulinum toxin, Bundle forming pilus structural subunit, C2 toxin, C3 toxin, C5A peptidase, Cardiotoxin, Chemotaxis, Cholera toxin, Ciliotoxin, Clostridial cytotoxin, Clostridial neutotoxin, Collagen adhesion gene, Crystal endotoxin, CyaA toxin, Cytolysin, Delta toxin, Delta toxin, Delta-lysin, Diphtheria toxin, Emetic toxin, Endotoxin, Staphylococcal Enterotoxins A, B, C1, C2, C3, D, E, G , Enterotoxin, Exfoliative toxin, Exotoxin, Exotoxin A, Exotoxin B, Exotoxin C, Extracellular elastase, Fibrinogen, Fibronectin binding protein i.e. fnbA, Filamentous hemagglutinin, Fimbriae, Gamma hemolysin, Gelatinase i.e.gelE, Haemolysin, Hemolysin B, Hemagglutinin, Histolyticolysin, IGG binding protein A i.e. spai, Intimin, Invasin, Iron siderophores, Ivanolysin, Ivanolysin O, Lantibiotic modifying enzyme, Lantibiotic structural protein, Lecithinase, Ler (positive regulator of LEE genes), Leukotoxin, Lipoprotein signal peptidase, Listeriolysin O, M protein, Motility, Neurotoxin, Nonfimbrial adhesins, Oedema factor, Perfringolysin O, Permease, Pertussis toxin, Phospholipase, Pili, Plasmid encoded regulator per, Pneumolysin, Poly-D-glutamic acid capsule, Pore-forming toxin, Proline permease, RNAIII, RTX toxin, Serine protease, Shiga toxinm, Siderophore/iron acquisition protein, SigA proteases, Spe A, Spe B, Spe C, STa toxin, Stb toxin, Streptolysin O, Streptolysin S, Superantigen, Superoxide dismutase, TCP, Tetanus toxin, Thiol-activated cytolysin, Tracheal cytotoxin, TSST toxin (TSST-1), and Urease, Zona occludens toxin.

37. The method of claim 33, wherein the bacterial cell is a pathogenic bacterial cell.

38. The method of claim 33, wherein the bacterial cell is selected from the group consisting of Vibrio harveyi, Vibrio cholerae, Vibrio parahaemolyticus, Vibrio alginolyticus, Pseudomonas phosphoreum, Yersinia enterocolitica, Escherichia coli, Salmonella typhimurium, Haemophilus influenzae, Helicobacter pylori, Bacillus subtilis, Borrelia burgfdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes and Staphylococcus aureus.

39. A method for regulating expression of a siderophore polypeptide in a bacterial cell comprising contacting a bacterial cell capable of producing the siderophore polypeptide with a compound identified by the method of claim 12, claim 26 or claim 29.

40. The method of claim 39, wherein the regulating is inhibition of expression of the siderphore polypeptide.

41. The method of claim 39, wherein the bacterial cell is a pathogenic bacterial cell.

42. The method of claim 39, wherein the bacterial cell is selected from the group consisting of Vibrio harveyi, Vibrio cholerae, Vibrio parahaemolyticus, Vibrio alginolyticus, Pseudomonas phosphoreum, Yersinia enterocolitica, Escherichia coli, Salmonella typhimurium, Haemophilus influenzae, Helicobacter pylori, Bacillus subtilis, Borrelia burgfdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes and Staphylococcus aureus.

43. A method for regulating bacterial colony morphology comprising contacting a bacterial colony with a compound identified by the method of claim 13, claim 29 or claim 32.

44. The method of claim 43, wherein the regulating is inhibition of smooth colony morphology formation.

45. The method of claim 43, wherein the bacterial colony contains pathogenic bacterial cells.

46. The method of claim 45, wherein the bacterial cells are selected from the group consisting of Vibrio harveyi, Vibrio cholerae, Vibrio parahaemolyticus, Vibrio alginolyticus, Pseudomonas phosphoreum, Yersinia enterocolitica, Escherichia coli, Salmonella typhimurium, Haemophilus influenzae, Helicobacter pylori, Bacillus subtilis, Borrelia burgfdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes and Staphylococcus aureus.

47. A method for treating a subject infected with a pathogenic bacterial cell comprising administering to the subject an inhibitor or antagonist that regulates LuxO binding to σ⁵⁴.

48. A method for inhibiting bacterial cell growth or virulence in a subject, comprising administering to the subject an inhibitor or antagonist that regulates LuxO binding to σ⁵⁴.

49. The method of claim 48, further comprising killing the bacterial cell after inhibiting its growth.

50. The method of claim 49, wherein the bacterial cell is killed by administering an antibiotic agent.

51. The method of claim 49, wherein the bacterial cell is killed by action of the immune system of the patient.

52. A bacterial cell comprising a distinct alteration in the rpoN gene, wherein the alteration results in an rpoN⁻ phenotype.

53. The cell of claim 52, wherein the bacterial cell is Vibrio harveyi rpoN::Cm^{r}.
